(19) United States
(12) Patent Application Publication
(10) Pub. No.: US 2007/0003607 A1
(43) Pub. Date: Jan. 4, 2007

(54) Neutral liposome-encapsulated compounds and methods of making and using thereof

(76) Inventors: Vibhudutta Awasthi, San Antonio, TX (US); William T. Phillips, San Antonio, TX (US); Beth A. Goins, San Antonio, TX (US)

Correspondence Address: NEEDLE & ROSENBERG, P.C., SUITE 1000, 999 PEACHTREE STREET, ATLANTA, GA, 30309-3915, US

(21) Appl. No.: 10/525,892

(22) PCT Filed: Aug. 27, 2004
(86) PCT No.: PCT/US04/27880

§ 371 Date: May 16, 2005

Related U.S. Application Data
(60) Provisional application No. 60/499,850, filed on Sep. 2, 2003.

(51) Int. Cl. A61K 9/127; C12N 15/88

(52) U.S. Cl. 424/450; 435/458

(57) ABSTRACT

The disclosed matter relates to neutral liposomes containing an encapsulated compound and post-insertion compound. Also described herein are methods of making and using the neutral liposomes.

### CROSS-REFERENCE TO RELATED APPLICATIONS

This application claims benefit of priority from U.S. Provisional Application No. 60/499,850, filed Sep. 2, 2003, which is herein incorporated by reference in its entirety.

### STATEMENT OF FEDERALLY SPONSORED RESEARCH

This invention was made with government support under Grant N00014-00-1-0793 awarded by the Office of Naval Research. The government has certain rights in the invention.

### BACKGROUND

Liposomes are unilamellar or multilamellar lipid vesicles that enclose a three-dimensional space. The membranes of liposomes are formed by a bilayer of one or more lipid components having polar heads and non-polar tails. In an aqueous (or polar) solution, the polar heads of one layer orient outwardly to extend into the aqueous, or polar, solution and to form a continuous, outer surface. Unilamellar liposomes have one such bilayer, whereas multilamellar liposomes generally have a plurality of substantially concentric bilayers.

Liposomes are well recognized as being useful for encapsulating therapeutic agents, such as cytotoxic drugs or other macromolecules capable of modifying cell behavior, and carrying these agents to in vivo sites. Further, liposomes have also been used in vitro as valuable tools to introduce various chemicals, biochemicals, genetic material and the like into viable cells and biological systems, and as diagnostic agents.

One area that has recently been examined is the use of liposome-encapsulated hemoglobin (LEH) as an oxygen carrier that mimics membrane enclosed cellular structure of red cells (Rudolph, “Encapsulation of Hemoglobin in Liposomes,” in Blood substitutes: Physiological Basis of Efficacy, Intaglietta M. ed., pp 90-104, Birkhauser, Boston (1995); Sakai et al., Biotechnol. Prog., 12, 119-125, 1996; Phillips et al., J. Pharmacol. Exp. Ther., 288, 665-670, 1999). Free hemoglobin has low oxygen carrying capacity and is rapidly eliminated from the body, while polymerized or crosslinked hemoglobins are plagued with cytotoxicity and constriction of blood vessels due to their NO-scavenging activity (Reiss, Chem. Reviews, 101, 2797-2919, 2001; Squires, Science, 295, 1002-1005, 2002). The spatial isolation of hemoglobin by an oxygen permeable lipid bilayer in liposomes can eliminate the toxicity associated with free hemoglobin. In addition, with co-encapsulation of reductants, antioxidative enzymes, and oxygen-affinity modifiers it is possible to enhance resuscitative capacity of LEH. Despite these desirable properties, a major impediment in the development of LEH has been a low encapsulation efficiency of the hemoglobin inside the liposome.

To increase the encapsulation of proteins inside liposomes, anionic lipids, such as dimyristoyl- and dipalmitoyl-phosphatidyl glycerol (DMPG and DPPG) have been incorporated in the lipid composition (Drummond et al., Pharmacol. Rev., 51, 691-743, 1999; Walde et al., Biomol. Eng., 18, 143-177, 2001). However, anionic liposomes after intraveneous injection can rapidly interact with a biological system subsequent to their opsonization with complement and other circulating proteins (Harashima et al., Adv. Drug Delivery Rev., 32, 61-79, 1998; Miller et al., Biochemistry, 37, 12875-12883, 1998; Semple et al., Adv. Drug Delivery Rev., 32, 3-17, 1998; Szebeni, Crit. Rev. Ther. Drug Carrier Syst., 15, 57-88, 1998). Such an interaction can have at least two acute consequences: (1) a rapid uptake by the reticuloendothelial system (RES) and (2) toxic effects, such as pseudoallergy that is manifested as vasoconstriction, pulmonary hypertension, dyspnea, drop in circulating platelets and leukocytes, etc. The situation can become more challenging when huge quantities of liposomes need to be administered, e.g., as in the case of resuscitative LEH. Thus, a conflict occurs between the necessity to encapsulate maximum amounts of a desired compound, for example, hemoglobin, in the least amount of lipid using anionic lipids and to keep the charge-associated undesirable effects in check.

Thus, what is lacking in the art are liposomes that can encapsulate a variety of different compounds with high efficiency and that are not toxic in biological systems. Described herein are compositions that satisfy this need.

### SUMMARY

In accordance with the purposes of the disclosed materials, compositions, and methods, as embodied and broadly described herein, in one aspect, the disclosed matter relates to neutral liposomes comprised of an encapsulated compound and post-insertion compound. Also described herein are methods of making and using the neutral liposomes.

Additional advantages of the disclosed materials, compositions, and methods will be set forth in part in the description which follows, and in part will be obvious from the description, or can be learned by practice of the subject matter described herein. The advantages of the disclosed materials, compositions, and methods will be realized and attained by means of the elements and combinations particularly pointed out in the appended claims. It is to be understood that both the foregoing general description and the following detailed description are exemplary and explanatory only and are not restrictive of the disclosed materials, compositions, and methods, as claimed.

### BRIEF DESCRIPTION OF THE DRAWINGS

The accompanying drawings, which are incorporated in and constitute a part of this specification, together with the description serve to explain the principles of the disclosed materials, compositions, and methods.

FIG. 1 is a schematic diagram showing a multilamellar vesicle (MLV) with PEGylated lipid in both layers, a unilamellar vesicle (ULV) with PEGylated lipid in both layers prepared by a conventional PEGylation technique, and a unilamellar vesicle with PEGylated lipid in the outer layer prepared by the post-insertion technique.

FIG. 2 is a schematic of a continuous process for manufacturing LEH.

FIG. 3 is a graph showing the biodistribution of anionic, PEG-anionic, neutral, and PEG-neutral ^{99m}Tc-LEHs, given in % ID/organ, in the blood, spleen, liver, and kidney of rabbits at 24 hours.

FIG. 4 is a compilation of scintigraphic images of rabbits 1 h and 24 h after injection with ^{99m}Tc-LEH-neutral (left panel) and PEG-neutral LEH (right panel).

FIG. 5 is a compilation of scintigraphic images of rabbits 1 h and 24 h after injection with ^{99m}Tc-LEH anionic (left panel) and PEG-anionic LEH (right panel).

FIG. 6 is a graph showing the quantitative analysis of scintiimages acquired at 1 h. Regions of interest were drawn around various organs (heart, spleen, liver) and normalized with the total counts in the image.

FIG. 7 is a graph showing the circulation kinetics of ^{99m}Tc-LEH in rabbits. An aliquot of arterial blood was sampled at various times for radioactivity counting after injecting radiolabeled preparations. The amount of radioactivity at any particular time is given in terms of percent of radioactivity present in a sample withdrawn immediately after LEH injection.

FIG. 8 is a compilation of representative gamma camera rat (top panel) and rabbit (bottom panel) images acquired at 4 h, 24 h, and 48 h, after 25% exchange transfusion of ^{99m}Tc-LEH.

FIGS. 9a and 9b are graphs showing the accumulation of ^{99m}Tc-LEH in blood, spleen, liver, kidney, and lungs of both rats and rabbits at 48 h after 25% exchange transfusion. FIG. 9a shows accumulation based on % injected dose (ID) per organ and FIG. 9b shows accumulation based on % injected dose (ID) per gram of tissue.

FIG. 10 is a graph showing the circulation profiles of the LEH preparation in blood of both rats and rabbits at 48 h after 25% exchange transfusion.

FIG. 11 is a graph showing radioactivity counts of blood samples collected from rabbits after LEH injection. These counts represent circulating radiolabeled platelets at various times after LEH injection.

FIG. 12 is a graph showing automated complete blood cell counting of blood samples collected from rabbits after LEH injection. These values represent the circulating platelets at various times after LEH injection.

### DETAILED DESCRIPTION

The disclosed materials, compositions, and methods may be understood more readily by reference to the following detailed description of specific aspects of the materials and methods and the Examples included therein and to the Figures and their previous and following description.

Before the present materials, compositions, and methods are disclosed and described, it is to be understood that they are not limited to specific synthetic methods, specific compositions, or to particular formulations, as such may, of course, vary. It is also to be understood that the terminology used herein is for the purpose of describing particular aspects of the disclosed materials, compositions, and methods only and is not intended to be limiting.

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a neutral liposome” includes mixtures of two or more such liposomes, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Reference will now be made in detail to certain aspects described herein, examples of which are illustrated in the accompanying drawings.

Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a post-insertion compound is disclosed and discussed and a number of modifications that can be made to a number of hydrophilic compounds and/or anchoring compounds are discussed, each and every combination and permutation of the post-insertion compound and the modifications to its hydrophilic compound and/or anchoring compound that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Neutral Liposome

In one aspect, described herein are neutral liposomes comprising an encapsulated compound and a post-insertion compound, wherein the post-insertion compound comprises a hydrophilic component and an anchoring component, wherein the encapsulated compound is located within the neutral liposome, and the post-insertion compound is adjacent to the outer surface of the neutral liposome.

In one aspect, the neutral liposomes are composed of a combination of phospholipids and cholesterol as described in the following paragraphs. The charge or neutrality of the liposomes is defined by the Zeta potential carried by the liposome particles. In one aspect, a neutral liposome is defined as liposome with a zeta potential ranging from zero to −20 meV based on the published research by Levchenko, et al., (Int. J. Pharma. 240: 95, 2002), which is incorporated by reference in its entirety.

The preparation of liposomes is well known in the art. The materials that can be used to prepare the neutral liposomes described herein include any of the materials or combinations thereof known to those skilled in the art as suitable for liposome preparation.

A neutral liposome suitable for use in the compositions and methods described herein can be composed of one or more neutral lipids. Such a neutral lipid is one that (1) can form spontaneously into bilayer vesicles in water, as exemplified by the phospholipids, or (2) is stably incorporated into lipid bilayers, with its hydrophobic moiety in contact with the interior, hydrophobic region of the bilayer membrane, and its head group moiety oriented toward the exterior, polar surface of the membrane. There are a variety of synthetic neutral lipids and naturally-occurring neutral lipids that can be useful in the compositions and methods described herein.

In one aspect, neutral lipids can include, but are not limited to, synthetic or natural phospholipids. Typically, though not required, a neutral lipid has two hydrocarbon chains, e.g., acyl chains, and either a polar, nonpolar, or zwitterionic head group. The two hydrocarbon chains can be any length. In one aspect, the hydrocarbon chain is between about 14 to about 22 carbon atoms in length, and can have varying degrees of unsaturation. In another aspect, the neutral lipid has a high molecular weight and high melting temperature.

Neutral lipids which can be used to create neutral liposomes include, but are not limited to, phosphatidylcholine (PC), phosphatidylethanolamine (PE), sphingomyelin (SPM), distearoylphosphatidylcholine (DSPC), dipalmitoylphosphatidylcholine (DPPC), dimyristoylphosphatidylcholine (DMPC), diarachidonoylphosphatidylcholine (DAPC), egg phosphatidylcholine, hydrogenated soy phosphatidylcholine (HSPC), glycosphingolipids and glycoglycerolipids, and sterols such as cholesterol, either alone or in combination with other lipids. In one aspect, the neutral lipid is distearoylphosphatidylcholine. Such neutral lipids can be obtained commercially or can be prepared by methods known to one of ordinary skill in the art.

In one aspect, one or more, anionic lipids can optionally be used to produce the neutral liposomes disclosed herein, where the anionic lipid can be included as a minor component of the lipid composition. Not wishing to be bound by theory, it is believed that the anionic lipid (1) makes the liposome more stable because the negative charge repels other liposomes; and (2) the negative charge makes the liposome more interactive with other molecules present in the subject. Such anionic lipids can have a lipophilic moiety, such as a sterol, an acyl chain, or a diacyl chain, and where the lipid has an overall net negative charge. The head group of an anionic lipid typically carries the negative charge. Typically, though not required, the lipophilic moiety of an anionic lipid contains two hydrocarbon chains, which can be any length. In one aspect, the hydrocarbon chain is from about 14 to about 22 carbon atoms in length, and can have varying degrees of unsaturation. In another aspect, the anionic lipid has a high molecular weight and high melting temperature.

Suitable anionic lipids include, but are not limited to, phospholipids that contain phosphatidylglycerol, phosphatidylserine or phosphatidic acid headgroups and two saturated fatty acid chains containing from about 14 to about 22 carbon atoms. Other suitable anionic lipids include, but are not limited to, phosphatidylserine (PS), phosphatidylglycerol (PG), phosphatidic acid (PA), phosphatidylinositol (PI), cardiolipin, dimyristoylphosphatidylglycerol (DMPG), and dipalmitoylphosphatidylglycerol (DPPG). In one aspect, the anionic lipid is dimyristoylphosphatidylglycerol. Such anionic lipids can be obtained commercially or can be prepared by methods known to one of ordinary skill in the art.

In one aspect, the total amount of the anionic lipid in the neutral liposome can be less than about 6 mole percent of the total lipids. In another aspect, the amount of anionic lipid is from about 6% to about 0.1%, about 5% to about 0.5%, about 4% to about 1%, or about 3% to about 1.5%, by molar ratio of the total lipids.

In one aspect, lipids having phase transition temperatures (T_{c}) from about 2° C. to about 80° C. are suitable for use in preparing the neutral liposomes described herein. In one aspect, lipids with elevated transition temperatures, such as DSPC (T_{c} of about 55° C.), DPPC (T_{c} of about 41° C.), and DAPC (T_{c} of about 66° C.), are heated to about their T_{c} or temperatures slightly higher, e.g., up to about 5° C. higher than the T_{c}, in order to make neutral liposomes. Phase transition temperatures of many lipids are tabulated in a variety of sources, such as Avanti Polar Lipids catalogue and Lipid Thermotropic Phase Transition Database (LIPIDAT, NIST Standard Reference Database 34), which is incorporated herein by reference in its entirety.

The lipids used to prepare the neutral liposomes described herein can be chosen by one of ordinary skill in the art based upon the particular conditions, uses, and purposes of the neutral liposome. For example, the lipids can be selected to achieve a specified degree of fluidity or rigidity, to control the stability of the neutral liposome in serum, to control the conditions effective for insertion of the encapsulated compound or post-insertion compound, as will be described, and to control the rate of release of the encapsulated compound from the neutral liposome. The lipids may be of synthetic as well as natural origin.

Neutral liposomes having a more rigid lipid bilayer can be achieved by incorporation of a relatively rigid lipid, e.g., a lipid having a relatively high phase transition temperature, e.g., up to about 60° C. Rigid, i.e., saturated, lipids contribute to greater membrane rigidity in the lipid bilayer. Other components, such as cholesterol, are also known to contribute to membrane rigidity in lipid bilayer structures. On the other hand, lipid fluidity is achieved by incorporation of a relatively fluid lipid, typically one having a relatively low gel to liquid-crystalline phase transition temperature, e.g., at or below room temperature.

The size of the neutral liposomes can be adjusted, if desired, by a variety of procedures including extrusion, filtration, sonication, homogenization, microfluidization employing a laminar stream of a core of liquid introduced into an immiscible sheath of liquid, extrusion under pressure through pores of defined size, and similar methods, in order to modulate resultant liposomal biodistribution and clearance. The foregoing techniques, as well as others, are discussed, for example, in Mayer et al., Biochim. Biophys. Acta, 858, 161-168, 1986; Hope et al., Biochim. Biophys. Acta, 812, 55-65, 1985; Mayhew et al., Methods in Enzymology, 149, 64-77, 1987. The disclosures of the foregoing publications are incorporated by reference herein in their entirety. The size of the liposome can be important in some situations; for example, large liposomes can be rapidly eliminated from circulation by phagocytic cells of RES (Allen et al., Biochim. Biophys. Acta, 1068, 122-141, 1991).

In one aspect, the size of the neutral liposome can be from about 100 nm to about 350 nm. The size of the neutral liposome can be about 100 nm, 125 nm, 150 nm, 175 nm, 200 nm, 225 nm, 250 nm, 275 nm, 300 nm, 325 nm, or 350 nm, where any of the stated values can form an upper and/or lower endpoint when appropriate. In yet another aspect, the size of the neutral liposome is from 210 nm to 240 nm. In still another aspect, the neutral liposome has a uniform size distribution.

The neutral liposome can further include a steroid compound, an antioxidant, or a combination thereof. That is, any of the above-mentioned liposome-forming lipids can be used in combination with at least one additional component such as a steroid or antioxidant.

While not wishing to be bound by theory, steroid compounds are believed to impart strength to the neutral liposome by making the lipid bilayer more rigid and the liposome less likely to leak the encapsulated compounds. Suitable steroids that can be used with the neutral liposomes described herein include, but are not limited to, cholesterol, cholestanol, coprostanol or cholestane. In addition, polyethylene glycol derivatives of cholesterol (PEG-cholesterols), as well as organic acid derivatives of sterols, e.g., cholesterol hemisuccinate (CHS), can also be used in combination with any of the above-mentioned lipids. Organic acid derivatives of α-tocopherol hemisuccinate (THS) can also be used. CHS- and THS-containing neutral liposomes and their tris salt forms can generally be prepared by methods known in the art for preparing liposomes containing sterols, so long as the resultant phospholipid-sterol mixture yields stable liposomes. In one aspect, the steroid is cholesterol. Such steroid compounds can be obtained commercially or can be prepared by methods known to one of ordinary skill in the art. The amount of steroid that can be incorporated into the liposome will vary depending upon the nature of the liposome, the encapsulated compound, and the application of the neutral liposome.

In one aspect, the amount of steroid that can be incorporated into the liposome can be from about 0 to about 50 mole % or from about 30 to about 50 mole %. In another aspect, the amount of steroid that can be incorporated into the liposome can be about 0, 5, 10, 15, 20, 25, 30, 35, 40, 45, 50, 55, or 60 mole %, where any of the stated values can form an upper and/or lower endpoint when appropriate.

In any of the methods for making the neutral liposomes described herein, an antioxidant can optionally be incorporated within the liposome. The procedures for incorporating an antioxidant into a liposome disclosed in U.S. Pat. Nos. 5,143,713 and 5,158,760, which are incorporated by reference in their entireties, can be used. In another aspect, the neutral liposome can be contacted with the antioxidant after preparation.

The use of antioxidants can be particularly beneficial when the neutral liposome encapsulates a compound that is sensitive to oxidation such as, for example, hemoglobin. Hemoglobin contains four heme groups, and when the heme iron is ferrous iron (Fe²⁺), oxygen can be reversibly bound. However, when the heme iron is ferric iron (Fe³⁺) (called methemoglobin), oxygen cannot bind. In addition, oxygen-bound hemoglobin gradually releases a superoxide anion and changes into methemoglobin. Furthermore, the superoxide anion acts as an oxidizing agent to accelerate production of methemoglobin. In erythrocytes, there is a methemoglobin reducing system and an active free radical removal system. These systems prevent the content of methemoglobin from increasing, whereas, in the liposomes with encapsulated hemoglobin, these enzymatic systems are not typically present. Therefore, hemoglobin may be oxidized during storage and after administration (to a subject), lowering the oxygen-carrying ability. To suppress the oxidation reaction, a mild antioxidant such as, for example, glutathione or homocysteine, can be included in the liposome encapsulated hemoglobin. With the inclusion of such antioxidants, heme iron that has been previously oxidized into ferric iron is reduced to ferrous iron.

Suitable antioxidants that can be used with the disclosed neutral liposomes can be water soluble or soluble in an organic solvent. Specific examples of water-soluble antioxidants include, but are not limited to, ascorbic acid, glutathione, and homocysteine. Specific examples of lipophilic antioxidants include, but are not limited to, tocopherol analogues, namely vitamin E. There are four isomers of tocopherol, α, β, γ, δ, each of which is useful in the neutral liposomes described herein. In one aspect, the antioxidant is α-tocopherol. Such antioxidants can be obtained commercially or can be prepared by methods known to one of ordinary skill in the art.

In one aspect, the antioxidant can be used with the neutral liposomes in an amount of from about 0.5 to about 4.5 mole percent, or from about 1.0 to about 2.0 mole percent per total amount of the lipid. In another aspect, the antioxidant can be used in an amount of from about 0.1, 0.5, 1.0, 1.5, 2.0, 2.5, 3.0, 3.5, 4.0, 4.5, 5.0, or 5.5 mole percent per total amount of the lipid, where any of the values can form an upper and/or lower endpoint when appropriate.

Encapsulated Compound

As noted above, the neutral liposomes described herein contain an encapsulated compound located within the neutral liposome. The encapsulated compound can be located in the inner volume of the liposome and/or the encapsulated compound can be located within the membrane of the liposome. An unencapsulated compound can be the same kind of compound as an encapsulated compound, except that it is not located within the liposome, i.e., it is not located in the inner volume of the liposome and/or located inside the membrane of the liposome.

Suitable encapsulated compounds include, but are not limited to, hemoglobin, a protein, an enzyme, an immunoglobulin, a peptide, an oligonucleotide, or a nucleic acid.

Encapsulated enzymes that can be used with the neutral liposomes described herein include, but are not limited to, alkaline phosphatase, D-amino acid oxidase, δ-aminolevulinate dehydratase, α-amylase, amyloglucosidase, ascorbate oxidase, asparaginase, butyrylcholinesterase, catalase, carbonic anhydrase, chloroperoxidase, cholesterol esterase, chymosin, chymosin+NEUTRASE®, chymotrypsin, α-chymotrypsin, COROLASE PN®, cyprosin, dextranase, DNA photolyase, DNA-(apurinic or apyrimidinic site) lyase, DNA polymerase, DNase I, elastase, enzyme extract from Lactobacillus helveticus, FLAVOURZYME®, β-fructofuranosidase, β-galactosidase, β-glucosidase, glucocerbroside-β-glucosidase, glucose oxidase, glucose oxidase-insulin, glucose-6-phosphate-dehydrogenase, β-glucuronidase, hexokinase, β-lactamase, lipase from Chromobacterium viscosum, luciferase, lysozyme, NEUTRASE®, NEUTRASE®+phospholipase C, pepsin A, peroxidase, peroxidase+glucose oxidase, phosphatase, phospatase from Citrobacter, phospholipase A₂, phospholipase C, phospholipase D, phosphorylase, phosphotriesterase, t-plasminogen activator, polynucleotide phosphorylase, proteinase, proteinase K, Q_{β} replicase/MDV-I RNA, ribonuclease A, rulactine, Sn-glycerol-3-phosphate O-acyltransferase, sphingomylinase, streptokinase, superoxide dismutase, superoxide dismutase+catalase, trypsin, tyrosinase, urease, and urate oxidase. Any of the enzymes disclosed in Walde et al., Biomol. Eng., 18, 143-177, 2001; Corvo et al., Biochim. Biophys. Acta, 1564, 227-236, 2002, which are incorporated herein by reference for their teachings of encapsulated enzymes, can be used as an encapsulated compound. Suitable enzymes that can be used in accordance with the neutral liposomes described herein, including those disclosed above, can be obtained from commercial sources or prepared by methods known to one skilled in the art.

Encapsulated nucleic acids and nucleic acid sequences that can be used with the neutral liposomes described herein include, but are not limited to, nucleic acids isolated from viral, prokaryotic, eukaryotic, bacterial, plant, animal, mammal, and human sources. Other kinds of nucleic acids include, but are not limited to, antisense oligonucleotides, aptamers, primers, plasmids, catalytic nucleic acid molecules, e.g., ribozymes, triplex forming molecules, and antiangiogenic oligonucleotides. Further examples include recombinant DNA molecules that are incorporated into a vector, such as an autonomously replicating plasmid or virus, or that insert into the genomic DNA of a prokaryote or eukaryote, e.g., as a transgene or as a modified gene or DNA fragment introduced into the genome by homologous recombination or site-specific recombination, or that exist as separate molecules, e.g., a cDNA or a genomic or cDNA fragment produced by PCR, restriction endonuclease digestion, or chemical or in vitro synthesis. Useful nucleic acids can also include any recombinant DNA molecule that encodes any naturally- or non-naturally occurring polypeptide. Other nucleic acids include RNA, e.g., an mRNA molecule that is encoded by an isolated DNA molecule, or that is chemically synthesized. Additional nucleic acids and oligonucleotides that can be encapsulated into liposomes can be found in Fillion et al., Biochim. Biophis. Acta, 1515, 44-54, 2001, which is incorporated herein by reference for its teachings of encapsulated nucleic acids. Suitable nucleic acids that can be used in accordance with the neutral liposomes described herein can be obtained from commercial sources or prepared by methods known to one skilled in the art.

The terms “nucleic acid,” “nucleotide,” “oligonucleotide,” “DNA,” and “RNA” are known to one of ordinary skill in the art. Definitions of these terms are also found in the World Intellectual Property Organization (WIPO) Handbook on Industrial Property Information and Documentation, Standard ST.25: Standard for the Presentation of Nucleotide and Amino Acid Sequence Listings in Patent Applications (1998), including Tables 1 through 6 in Appendix 2, incorporated herein by reference (hereinafter “WIPO Standard ST.25 (1998)”). In certain aspects described herein, the terms “nucleic acid,” “DNA,” and “RNA” include derivatives and biologically functional equivalents. In certain aspects described herein, the terms “nucleic acid,” “nucleic acid sequence,” and “oligonucleotide” are used interchangeably. These terms refer to a polymer of nucleotides (dinucleotide and greater), including polymers of 2 to about 100 nucleotides in length, including polymers of about 101 to about 1,000 nucleotides in length, including polymers of about 1,001 to about 10,000 nucleotides in length, and including polymers of more than 10,000 nucleotides in length.

In another aspect, amino acids and amino acid sequences such as proteins and peptides can be used with the neutral liposomes described herein. Suitable proteins can include, but are not limited to, insulin and pepsin. Also, encapsulated proteins and peptides can include large molecular weight therapeutic peptides and proteins such as, for example, GLP-1, CCK, antimicrobial peptides, and antiangiogenics. Proteins, such as insulin, that can be incorporated into liposomes can be found in Kim et al., Int. J. Pharm., 180, 75-81, 1999, which is incorporated herein by reference for its teachings of encapsulated proteins and peptides. Suitable proteins or peptides that can be used in accordance with the neutral liposomes described herein can be obtained from commercial sources or prepared by methods known to one skilled in the art.

The terms “amino acid” and “amino acid sequence” are known to one of ordinary skill in the art. Definitions of these terms are also found in the WIPO Standard ST.25 (1998). In certain aspects described herein, the terms “amino acid” and “amino acid sequence” include derivatives, mimetics, and analogues including D- and L-amino acids which cannot be specifically defined in WIPO Standard ST.25 (1998). The terms “peptide” and “amino acid sequence” are used interchangeably herein and refer to any polymer of amino acids (dipeptide or greater) typically linked through peptide bonds. The terms “peptide” and “amino acid sequence” include oligopeptides, protein fragments, analogues, nuteins, and the like.

In one aspect, the encapsulated compound is hemoglobin. The hemoglobin can be CO-complexed hemoglobin, which is generally useful to stabilize hemoglobin during processing. In another aspect, the hemoglobin can be stroma-free hemoglobin. Stroma-free hemoglobin can be isolated from erythrocytes of any animal or human source. Other useful hemoglobins included, but are not limited to, chemically modified hemoglobins, polymerized hemoglobins, hemoglobin mutants from any animal or human source that are genetically engineered and grown in bacteria or yeast, hemoglobins from any animal or human source that are prepared in transgenic animals. Other types of hemoglobin that can be encapsulated can be found in Reiss, Chem. Reviews, 101, 2797-2919, 2001, and U.S. Pat. No. 5,770,560 to Fisher et al., which are incorporated by reference herein for their teachings of hemoglobin and hemoglobin products.

The amount of encapsulated compound that can be incorporated within the neutral liposome can be from about 1.0 to about 12 g/dl. However, the ultimate therapeutic formulation can contain a lower or higher concentration depending on therapeutic application. In one aspect, the amount of encapsulated compound that can be incorporated within the neutral liposome can be about 1, 2, 3, 4, 5, 6, 7, 8, 9, 10, or 12 g/dl, where any of the stated values can form an upper and/or lower endpoint when appropriate. In another aspect, the amount of encapsulated hemoglobin that can be incorporated within the neutral liposome is from about 2 to 10 g/dl.

Post-Insertion Compound

As noted above, the neutral liposomes described herein contain a post-insertion compound. The post-insertion compound is the reaction product between a hydrophilic compound and an anchoring compound. The resultant post-insertion compound has a hydrophilic component and an anchoring component. The hydrophilic component of the post-insertion compound provides a surface coating around the liposome that acts as a barrier to phagocytosis. The anchoring component of the post-insertion compound attaches the post-insertion compound to the outer surface of the liposome by incorporating into the membrane of the liposome. The post-insertion compound can be effective at increasing the in vivo blood circulation lifetime of the liposomes when compared to liposomes lacking such a coating, and it is effective at reducing aggregation of the liposomes.

The reaction between an anchoring compound and a hydrophilic compound has been described in, for example, U.S. Pat. Nos. 5,013,556, 5,395,619, and 6,316,028, as well as in Sou et al., Bioconj. Chem., 11, 372-379, 2000; Carrion et al., Chem. Phys. Lipids, 113, 97-110, 2001; Sriwongsitanont et al., Chem. Pharm. Bull., 50, 1238-1244, 2002; Ishiwata et al., Chem. Pharm. Bull., 46, 1907-1913, 1998; Yuda et al., Biol. Pharm. Bull., 19, 1347-1351, 1996; Vertut-Doi et al., Biochim. Biophys. Acta, 1278, 19-28, 1996; and Webb et al., Biochim. Biophys. Acta, 1372, 272-282, 1998, which are all incorporated herein by reference for their teachings of methods of derivatizing anchoring compounds with hydrophilic compounds.

Hydrophilic compounds that are suitable for use in the post-insertion compound include, but are not limited to, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethylene glycol, polyaspartamide, or a hydrophilic peptide sequence, including combinations and mixtures thereof. The hydrophilic compound can be employed as homopolymers or as block or random copolymers. Such hydrophilic compounds can be obtained commercially or can be prepared by methods known to one of ordinary skill in the art.

In one aspect, the hydrophilic compound can have a molecular weight between about 500 to about 20,000 daltons, about 1,000 to about 15,000 daltons, or about 5,000 to about 10,000 daltons. In another aspect, the hydrophilic compound can have a molecular weight of about 500, 2,000, 5,000, 6,000, 8,000, 10,000, 12,000, 15,000, 18,000, and 20,000 daltons, where any of the stated values can form an upper and/or lower endpoint when appropriate.

In one aspect, the hydrophilic compound is a polyether diol such as, for example, polyethylene glycol, polypropylene glycol, and polybutylene glycol. In another aspect, the polyether diol is polyethylene glycol (PEG), having a molecular weight between about 500 to about 20,000, about 1,000 to about 10,000, about 1,000 to 5,000, or about 500 to about 5,000 daltons. Methoxy or ethoxy-capped analogues of PEG can also be used and are commercially available in a variety of polymer sizes, e.g., from about 120 to about 20,000 daltons.

In one aspect, anchoring compounds that can be used to produce the post-insertion compound include, but are not limited to, any of those lipids listed above for the neutral liposome, and, in particular phospholipids, such as distearoyl phosphatidylethanolamine (DSPE). Specific anchoring compounds that are useful include, but are not limited to, phosphatidylethanolamine with fatty acid chains having from about 14 to about 22 carbon atoms, cholesterol, ceramide, distearoylphosphatidylcholine, monogalactosyl diacylglycerols, dipalmitoyl phosphatidylethanolamine, or digalactosyl diacylglycerols. Other suitable anchoring compounds can be found in Sou et al., Bioconj. Chem., 11, 372-379, 2000; Carrion et al., Chem. Phys. Lipids, 113, 97-110, 2001; Sriwongsitanont et al., Chem. Pharm. Bull., 50, 1238-1244, 2002; Ishiwata et al., Chem. Pharm. Bull., 46, 1907-1913, 1998; Yuda et al., Biol. Pharm. Bull., 19, 1347-1351, 1996; Vertut-Doi et al., Biochim. Biophys. Acta, 1278, 19-28, 1996; Webb et al., Biochim. Biophys. Acta, 1372, 272-282, 1998, which are all incorporated herein by reference for their teachings of anchoring compounds. Anchoring compounds are generally available commercially or can be prepared by methods known to one of ordinary skill in the art.

As noted above, the post-insertion compound is the reaction product between a hydrophilic compound and an anchoring compound. It is contemplated that any of the hydrophilic compounds discussed above can be reacted with any of the anchoring compounds discussed above to form a post-insertion compound. The choice of a particular post-insertion compound, including the choice of the hydrophilic compound and the choice of the anchoring compound, can be readily determined by one of ordinary skill in the art based upon factors such as the particular purpose of the neutral liposome, the particular encapsulated compound, the particular conditions under which the neutral liposome is to be used or exposed, and the like. In one aspect, the post-insertion compound is polyethylene glycol-distearoylphosphatidylethanolamine.

In one aspect, the post-insertion compound can be present in the neutral liposome formulation in an amount between about 0.025 to about 15 mole percent based on the total amount of lipid in the liposome. In another aspect, the post-insertion compound can be present in the neutral liposome formulation in an amount of about 0.025, 0.05, 0.1, 0.2, 0.5, 1, 2, 4, 5, 6, 8, 10, 12, 14, or 15 mole percent based on the total amount of lipid in the liposome, where any of the stated values can form an upper or lower endpoint when appropriate.

Plasma Expanders

The neutral liposomes described herein can optionally contain a plasma expander. Not wishing to be bound by theory, it is believed that the plasma expander helps maintain the size of the liposome and imparts oncotic pressure. Suitable plasma expanders include, but are not limited to, a starch compound, albumin, dextran, and gelatin. Other plasma expanders include, but are not limited to, substituted or unsubstituted pentastarch, hetastarch, and hydroxyethyl starch, either alone or in combination. The use of a plasma expander for liposomes is disclosed in Roberts and Bratton, Drugs, 55(5), 621-630, 1998, and U.S. Pat. Nos. 5,589,189 and 6,033,708, which are incorporated by reference herein for their teachings of plasma expanders and their use thereof. Plasma expanders are available commercially or can be prepared by methods known to one of ordinary skill in the art. The particular amount of plasma expander can be readily determined by one of ordinary skill in the art based upon factors such as the particular purpose of the neutral liposome, the particular encapsulated compound, the particular conditions under which the neutral liposome is to be used or exposed, and the like.

In one aspect, the plasma expander can be present in the neutral lipid in an amount of from about 1 to about 10%. In another aspect, the plasma expander can be present in the neutral lipid in an amount of from about 5 to about 10%. In still another aspect, the plasma expander can be present in the neutral lipid in an amount of about 1, 2, 3, 4, 5, 6, 7, 8, 9, or 10%, where any of the stated values can form an upper and/or lower endpoint when appropriate.

Methods of Encapsulating Components in Neutral Liposomes

Methods for the encapsulation of compounds into liposomes are known in the art. For example, methods suitable for encapsulating enzymes in the neutral liposomes described herein are disclosed in Walde et al., Biomol. Eng., 18, 143-177, 2001, which is incorporated herein for its teachings of enzyme-encapsulation methods. Also, methods suitable for encapsulating enzymes, such as superoxide dismutase, in the neutral liposomes described herein are disclosed in Corvo et al., Biochim. Biophys. Acta, 1564, 227-236, 2002, which is incorporated herein for its teachings of enzyme-encapsulation methods. Methods suitable for encapsulating proteins such as, for example, insulin in the neutral liposomes described herein are disclosed in Fillion et al., Biochim. Biophys. Acta, 1515, 44-54, 2001; Kim et al., Int. J. Pharm., 180, 75-81, 1999, which are incorporated herein by reference for their teachings of encapsulating methods. Methods suitable for encapsulating hemoglobin are disclosed in Reiss, Chem. Reviews, 101, 2797-2919, 2001, which is incorporated by reference herein for its teachings of hemoglobin and hemoglobin product encapsulating methods.

Described herein are methods for preparing neutral liposomes with encapsulated compounds. In one aspect, the method for preparing a liposome-encapsulated compound, involves (a) admixing an unencapsulated compound with at least one neutral lipid; (b) microfluidizing the suspension produced in step (a) to produce a mixture comprising a first liposome and unencapsulated compound; (c) ultrafiltering the mixture produced in step (b) to remove the unencapsulated compound; and (d) contacting the resultant liposomes after ultrafiltering step (c) with a post-insertion compound.

Any of the materials described above, e.g., liposomes, lipids, encapsulated compounds, unencapsulated compounds, and post-insertion compounds, can be used in any of the methods described herein.

Step (a) involves admixing an unencapsulated compound with at least one neutral lipid. The admixing step can be performed by methods known in the art. For example, admixing can be accomplished by simultaneously combining the unencapsulated compound and at least one neutral lipid together or by sequentially adding one to the other. Also, either one or both of the unencapsulated compound and at least one neutral lipid can be in dispersion when admixed or they can be neat when admixed and then later hydrated with an aqueous phase. Admixing can further involve stirring, shaking, or vortexing the admixture, which can be performed by, for example, a magnetic or mechanical stirrer or by a mechanical shaker, spinner, or tumbler. Admixing can further involve bubbling an inert gas though the admixture. Also, admixing can further involve sonication.

Step (b) involves microfluidizing the suspension provided after admixing. During microfluidization, a high pressure device such as a MICROFLUIDIZER™ is used. In microfluidization, a large amount of energy is imparted to the liposomes during the short period of time during which the fluid passes through a high pressure interaction chamber at, for example, from about 2,000 to about 4,000 psi. In the interaction chamber, two streams of fluid at a high speed collide with each other at about a 90° angle. As the microfluidization temperature increases, the fluidity of the membrane also increases, which initially makes particle size reduction easier, as expected. For example, filterability can increase by as much as four times with the initial few passes through a MICROFLUIDIZER™ device. The increase in the fluidity of the bilayer membrane promotes particle size reduction, which makes filtration of the final composition easier. The microfluidization techniques disclosed in U.S. Pat. Nos. 4,776,991 and 4,911,929, which are incorporated by reference in their entireties, can be used in the methods described herein. Microfluidization can be easily scaled up to industrial level.

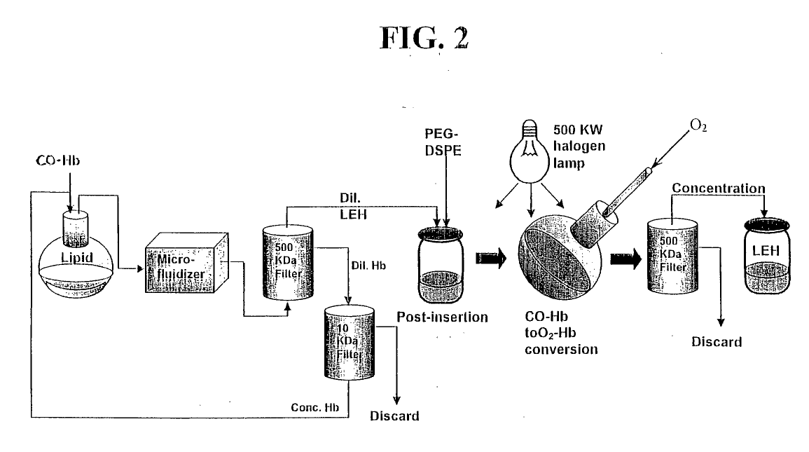

After microfluidization, the resultant mixture is ultrafiltered to remove all or the majority of the unencapsulated compound. In some instances, there can be unencapsulated compound from, for example, diffusion of encapsulated compound out of the liposome or from excess compounds that have not been encapsulated. In these situations, it may be desired to remove the unencapsulated compound from the liposome formulation. In other instances there may be no unencapsulated compound, i.e., 100% encapsulation efficiency. Generally, though, the unencapsulated compound can be removed by various means such as dialysis, centrifugation, filtration, sedimentation and column chromatography. In one aspect, ultrafiltration using filter cartridges of defined molecular weight cutoff is used. Suitable ultrafiltration cartridges are commercially available from Amersham Biosciences, Millipore or other vendors. One set of cartridges of various molecular size cutoffs is shown in FIG. 2.

After the neutral liposomes with the encapsulated compound have been prepared, it is desirable to impart greater stability to the liposome in a biological environment. One way to inhibit or reduce the physiological response to a liposome is to conceal the liposome surface with hydrophilic component by incorporating a lipid derivatized hydrophilic compound such as poly(ethylene glycol)-phosphatidylethanolamine (PEG-PE) into the bilayer structure. Inclusion of such derivatized lipids can improve storage stability, reduce RES uptake, and decrease dependence on small size to achieve prolonged circulation of liposomes. While not wishing to be bound by theory, it is believed that with the derivatized lipid, the hydrophilic component coats the liposome surface to create a steric barrier, enabling liposomes to circulate longer. Secondary to the steric hindrance, inhibition of liposome-induced complement activation can also be partially responsible for the beneficial effects of such derivatized lipids like PEG-PE (Ahl et al., Biochim. Biophys. Acta, 1329, 370-382, 1997; Devine and Bradley, Devine et al., Adv. Drug Delivery Rev., 32, 19-29, 1998).

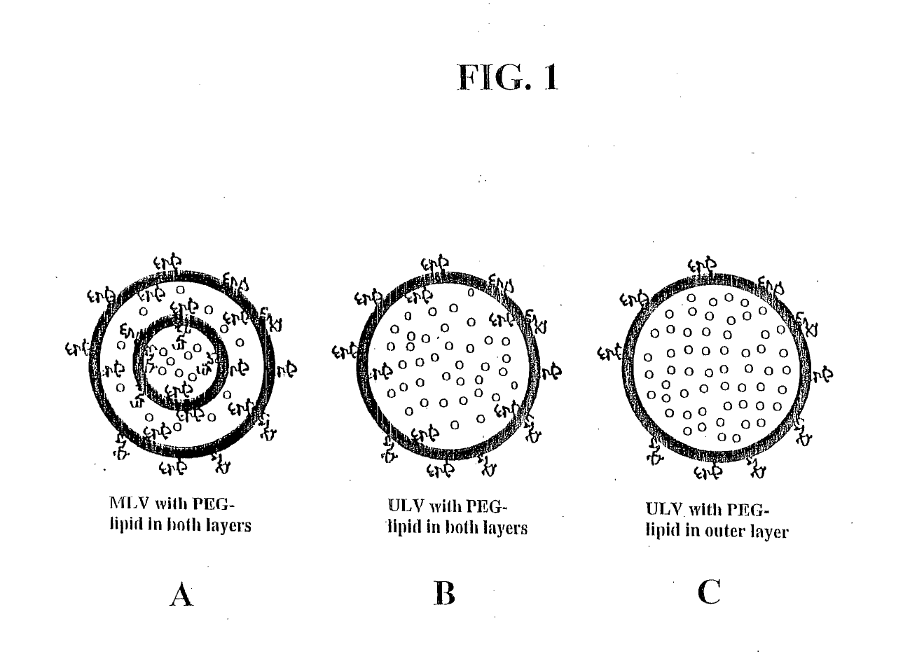

Incorporation of a lipid derivatized with a hydrophilic compound in the liposome bilayer can be done when preparing lipid phase just prior to its hydration with an aqueous phase. However, as illustrated in FIG. 1, this technique can result in the hydrophilic compound portion of the derivatized lipid to be associated with both layers of the bilayer membrane, and thus the hydrophilic compound portion occupies the space inside the liposomes. Theoretically, in a liposome with size of 200 nm and a hydrophilic compound portion of 5 nm, there is a net reduction of ˜15% space available for the encapsulated compounds. The smaller the size or the greater the lamellarity of liposomes, the greater is the impact of the hydrophilic compound on total usable space for encapsulated material (FIG. 1). Furthermore, this technique requires more derivatized lipid than is needed for useful stealthing of a liposome; thus underutilizing expensive lipid. In the case of multilamellar liposomes, the magnitude of wastage is more, because inner bilayers do not materially contribute to the in vivo behavior of the liposomes. In addition, the same steric hindrance that helps enhance circulation in vivo may inhibit the encapsulation of substances by exclusion phenomenon (Nicholas et al., Biochim. Biophys. Acta, 1463, 167-178, 2000). This exclusion reduces the encapsulation efficiency, especially of macromolecules, such as hemoglobin. Although techniques where a post-insertion compound having an anchoring component and a hydrophilic component is inserted in the outer layer of liposomes (FIG. 1) after final manufacturing stages have been developed (see Uster et al., FEBS Lett., 386, 243-246, 1996; Sakai et al., Bioconj. Chem., 8, 23-30, 1997; Sakai et al., Bioconj. Chem., 11, 425-432, 2000), these post-insertion methods were not developed specifically for the purpose of increasing encapsulation efficiency, and these prior studies have not reduced to practice the use of a post-insertion method for increasing encapsulation efficiency. These prior post-insertion studies used liposomes comprising 10% or greater negatively charged lipids, and differ significantly from the non-toxic neutral lipids disclosed herein.

In one aspect, after ultrafiltration, the liposomes are contacted with a post-insertion compound. Contacting can be accomplished by means known in the art and involves introducing the post-insertion compound to the liposome formulation. The result of this contacting step is that the post-insertion compound becomes adjacent to the outer surface of the liposome. As used herein “adjacent to the outer surface” refers to instances where the anchoring component of the post-insertion compound is inserted into the outer lipid layer of the liposome and the hydrophilic component of the post-insertion compound extends out over the liposome's outer surface and is therefore next to or near the outer surface. Also, “adjacent to the outer surface” includes instances where the post-insertion compound has not inserted into the lipid bilayer of the liposome, or has only partially inserted into the bilayer, and the hydrophilic component of the post-insertion compound is next to or near the outer surface of the liposome bilayer, as would be the result of intermolecular attractive forces.

The mechanistic basis for the interaction of the post-insertion compound with the liposome is discussed in Sou et al., Bioconj. Chem., 11, 372-379, 2000. While not wishing to be bound by theory, the amphiphilic post-insertion compound exists as a monomer below its critical micelle concentration and intercalates into the outer lipid layer of the liposome. The degree of incorporation is a function of the hydrophilic component length, anchoring component length, temperature, and concentration of lipids.

In one aspect, the neutral liposome can be contacted with a plasma expander. Methods for contacting liposomes with plasma expanders are known in the art and are described, for example, in Roberts et al., Drugs, 55, 612-630, 1998, which is incorporated by reference herein in its entirety.

In one aspect, a plasma expander can optionally be added after step (a), the admixing step, and prior to step (d), the contacting step. In another aspect, the plasma expander can be added after step (b), the microfluidizing step, and prior to step (c), the ultrafiltering step. The addition of a plasma expander, such as, for example, pentastarch, can be used to control the particle size distribution of the liposome after microfluidization. In one aspect, about 10 to about 25 mg/ml of a plasma expander can be added immediately after microfluidization of the liposome in order to maintain the size of the liposome within a narrow range of distribution. The size of the liposomes can be important, for example, with respect to pseudoallergic reaction that may ensue after an intravenous infusion of the liposomes.

In one aspect, when the neutral liposomes are prepared by a continuous process, the unencapsulated compound that has been removed can also be recycled. That is, the unencapsulated compound can be isolated and stored for later use or the unencapsulated compound can be admixed again with neutral lipid. A schematic of one aspect of the recycling process is shown in FIG. 2. In the first step, lipid is mixed with aqueous phase containing concentrated hemoglobin solution to generate a homogeneous suspension. The mixture is introduced into a microfluidizer to reduce the particle size of LEH before filtering (500 KDa MWCO) off unencapsulated hemoglobin. Unencapsulated hemoglobin is concentrated again by ultrafiltration (10 KDa MWCO) and re-introduced in the first step of the next cycle. The LEH preparation is taken for post-inserting PEG-DSPE at from about 25 to about 55° C., or about 37° C.

In one aspect, when the encapsulated compound is hemoglobin in carbonyl form, the hemoglobin can be converted to the oxy form. Referring to FIG. 2, hemoglobin in the PEG-LEH is converted from carbonyl form to oxy form by exposure to light (e.g., 500 W halogen lamp) and oxygen saturation. This is followed by concentration of dilute LEH by ultrafiltration (500 KDa MWCO).

Uses

The disclosed neutral liposomes with encapsulated compounds and post-insertion compounds have many uses. For example, when the neutral liposomes contain encapsulated hemoglobin, there can be wide spread applications in surgery, trauma, war-like situations, and any condition where blood transfusion is quickly required, but not available for the reasons of incompatibility, remote location, or the exhausted supply of blood. One advantage of using LEH is that it could minimize the risk of spread of infectious diseases by transfusion of blood contaminated with HIV, HBV, WNV, malarial parasite, etc. Also, neutral liposomes with encapsulated hemoglobin do not induce vasoconstriction by NO scavenging because the hemoglobin is encapsulated within the liposome. It is also possible with the neutral liposomes described herein to modulate oxygen affinity and in vivo stability of hemoglobin by co-encapsulating other substances.

In one aspect, disclosed herein is a method of treating or preventing a disease in a subject comprising administering to the subject a neutral liposome as discussed above. The selection of the encapsulated compound will determine if the neutral liposome can treat a disease in a subject. Reiss, Chem. Reviews, 101, 2849-2919, 2001, which is incorporated by reference in its entirety, discusses therapeutic uses of liposome-encapsulated hemoglobin. Also, liposomes with encapsulated antisense oligonucleotides have been used to combat bacterial infections (Fillon et al., Biochim. Biophys. Acta, 1515, 44-54, 2001). Liposomes with encapsulated superoxide dismutase have been used as a treatment to alleviate arthritis (Corvo et al., Biochim. Biophys. Acta, 1564, 227-236, 2002). Liposomes with encapsulated enzymes have been used for the treatment of myocardial infarction (Storm et al., J. Control Release, 36, 19-24, 1995). Other therapeutic uses of liposomes with encapsulated compounds are listed in Walde et al., Biomol. Eng., 18, 143-177, 2001, which is incorporated by reference herein in its entirety.

The dosage or amount of neutral liposome should be large enough to produce the desired effect in which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician in the event of any counterindications. The dose, schedule of doses and route of administration can be varied, whether oral, nasal, vaginal, rectal, extraocular, intramuscular, intracutaneous, subcutaneous, intravenous, intratumoral, intrapleural, intraperitoneal or other practical routes of administration to avoid adverse reactions yet still achieve delivery.

The neutral liposomes described herein can be used therapeutically in combination with a pharmaceutically acceptable carrier to produce a pharmaceutical composition. Pharmaceutical carriers are known to those skilled in the art. These most typically would be standard carriers for administration of compositions to humans and non-humans, including solutions such as sterile water, saline, and buffered solutions at physiological pH. In one aspect, the pharmaceutical compositions can include carriers, thickeners, diluents, buffers, preservatives, surface active agents and the like in addition to the molecule of choice. In one aspect, the pharmaceutical compositions described herein can be administered by injection including, but not limited to, intramuscular, subcutaneous, intraperitoneal, intratumoral or intraveneous injection. Other compounds will be administered according to standard procedures used by those skilled in the art.

In one aspect, the neutral liposomes described herein are administered to a subject such as a human or an animal including, but not limited to, a rodent, dog, cat, horse, bovine, ovine, or non-human primate and the like, that is in need of alleviation or amelioration from a recognized medical condition. The neutral liposomes can be administered to the subject in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The neutral liposomes described herein can be administered intravenously, intraperitoneally, intramuscularly, subcutaneously, intratumoral, intracavity, or transdermally.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

In another aspect, disclosed herein are methods for screening a liposome-encapsulated compound for an activity by (a) measuring a known activity or pharmacological activity of the liposome-encapsulated compound; and (b) measuring the same activity or pharmacological activity of the corresponding unencapsulated compound.

The activities for which the liposome-encapsulated compound can be screened can include any activity associated with a biologically active compound. The following is a partial list of the many activities that can be determined in the present screening method:

1. Receptor Agonist/Antagonist Activity:

A compendia of examples of specific screens for measuring these activities can be found in: “The RBI Handbook of Receptor Classification and Signal Transduction” K. J. Watling, J. W. Kebebian, J. L. Neumeyer, eds. Research Biochemicals International, Natick, Mass., 1995, and references therein. Methods of analysis can be found in: T. Kenakin “Pharmacologic Analysis of Drug-Receptor Interactions” 2nd Ed. Raven Press, New York, 1993, and references therein.

2. Enzyme Inhibition:

A compendia of examples of specific screens for measuring these activities can be found in: H. Zollner “Handbook of Enzyme Inhibitors”, 2nd Ed. VCH Weinheim, FRG, 1989, and references therein.

3. Central Nervous System, Autonomic Nervous System (Cardiovascular and Gastrointestinal Tract), Antihistaminic, Anti-Inflammatory, Anaesthetic, Cytotoxic, and Antifertility Activities:

A compendia of examples of specific screens for measuring these activities can be found in: E. B. Thompson, “Drug Bioscreening: Drug Evaluation Techniques in Pharmacology,” VCH Publishers, New York, 1990, and references therein.

4. Anticancer Activities:

A compendia of examples of specific screens for measuring these activities can be found in: I. J. Fidler and R. J. White “Design of Models for Testing Cancer Therapeutic Agents,” Van Nostrand Reinhold Company, New York, 1982, and references therein.

5. Antibiotic and Antiviral (Especially Anti-HIV) Activities:

A compendia of examples of specific screens for measuring these activities can be found in: “Antibiotics in Laboratory Medicine,” 3rd Ed., V. Lorian, ed. Williams and Wilkens, Baltimore, 1991, and references therein. A compendia of anti-HIV screens for measuring these activities can be found in: “HIV Volume 2: Biochemistry, Molecular Biology and Drug Discovery,” J. Karn, ed., IRL Press, Oxford, 1995, and references therein.

6. Immunomodulatory Activity:

A compendia of examples of specific screens for measuring these activities can be found in: V. St. Georgiev, “Immunomodulatory Activity of Small Peptides,” Trends Pharm. Sci. 11, 373-378 1990.

7. Pharmacokinetic Properties:

The pharmacological activities assayed in the screening method include half-life, solubility, or stability, among others. For example, methods of analysis and measurement of pharmacokinetic properties can be found in: J.-P. Labaune “Handbook of Pharmacokinetics: Toxicity Assessment of Chemicals,” Ellis Horwood Ltd., Chichester, 1989, and references therein.

8. Oxygen Carrying Capacity

The functional capacity of compounds such as hemoglobin is assessed both in vitro as well as in vivo. Methods of analysis are described in: Reiss, Chem. Rev., 101, 2797, 2001 and references therein; Rabinovici et al., Circulatory Shock, 32, 1, 1990; Methods Enzymol., Vols. 231 & 232; Proctor, J. Trauma, 54, S106, 2003 and references therein.

In the screening method, the liposome can be any of the neutral liposomes described herein. Also, the encapsulated compound, which corresponds to the unencapsulated compound, can be any of the encapsulated compounds described herein. Thus, in the screening method contemplated herein, any neutral liposome with an encapsulated compound, i.e., liposome-encapsulated compound, can be compared to the corresponding unencapsulated compound having a known activity to determine whether or not it has the same or similar activity at the same or different level. Depending on the specifics of how the measuring step is carried out, the present screening method can also be used to detect an activity exhibited by the unencapsulated compound of step b) that differs qualitatively from the activity of the encapsulated compound of step a). Also, the screening method can be used to detect and measure differences in the same or similar activity. Thus, the screening methods described herein take into account the situation in which the differences of the liposome-encapsulated compound significantly alter the biological activity of the unencapsulated compound.

### EXAMPLES

The following examples are put forth so as to provide those of ordinary skill in the art with a complete disclosure and description of how the compounds, compositions, and/or methods claimed herein are made and evaluated, and are intended to be purely exemplary of the disclosed materials and methods and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Materials

The phospholipids, distearoylphosphatidylcholine (DSPC), dimyristoylphosphatidylglycerol (DMPG) and poly(ethylene glycol)5000-distearoylphosphatidylethanolamine (PEG₅₀₀₀-DSPE) were obtained from Avanti Polar Lipids (Pelham, Ala.). Cholesterol (C) was purchased from Calbiochem (La Jolla, Calif.) and α-tocopherol was purchased from Aldrich (Waukegan, Ill.). Glutathione (GSH), octyl-β-glucoside (OBG), and pyridoxal-5′ phosphate (PLP) were from Sigma (St. Louis, Mo.). The radiopharmaceutical, ^{99m}Tc-sodium pertechnetate, was obtained commercially (Amersham Health Nuclear Pharmacy, San Antonio, Tex.). For animal experiments, anesthetics xylazine and ketamine were from Phoenix Scientific, Inc. (St. Joseph, Mo.) and Fort Dodge Animal Health (Fort Dodge, Iowa).

Frozen human stroma-free oxy-hemoglobin (O₂-Hb) was carbonylated with carbon monoxide (CO) immediately after thawing the hemoglobin under aseptic conditions (Sakai et al., Bioconj. Chem., 8, 23-30, 1997) since carbonyl-hemoglobin (CO-Hb) is more stable than the O₂-Hb.

### Example 1

### Effect of PEG-DSPE Post-Insertion on Circulation Kinetics of Neutral and Anionic LEH

Preparation of LEH: Liposome encapsulated hemoglobin (LEH) (DSPC/Cholesterol/α-tocopherol, 51.4:46.4:2.2, and DSPC/Chol/DMPG/α-tocopherol, 46:42:9.8:2.2) were prepared by microfluidization technique. Briefly, a solution of lipids in chloroform was evaporated to a dry film in a rotary film evaporator (Brinkmann Instruments, NY). After further exposure of the lipid film to vacuum for 4-6 h, the dried lipid film was hydrated with a solution of sucrose (300 mM) in sterile water for injection. The suspension was lyophilized overnight and the dried mixture was again hydrated with 38% solution of CO-hemoglobin containing GSH (100 mM) and PLP (18 mM). The mixture was thoroughly mixed at room temperature to form a homogenous suspension and the particle size of the liposomes was reduced in a Microfluidizer (M110-T, Microfluidics Corp., Newton, Mass.). The bulk of the unencapsulated material was separated from LEH by tangential ultrafiltration through a 300 KDa cartridge (Millipore, Bedford, Mass.) using phosphate-buffered saline (PBS, pH 7.4) as the diluent. After filtration, the preparations were divided into two equal halves. One half of each preparation was PEGylated while the other half was further processed without PEGylation. For PEGylation, PEG₅₀₀₀-DSPE solution was added to a dilute suspension of LEH, such that the concentration of PEG₅₀₀₀-DSPE was below its critical micelle concentration (Sou et al., Bioconjug. Chem., 11, 372, 2000). The mixture was stirred for 1 h at 55° C. under CO atmosphere to enable insertion. The insertion of PEG₅₀₀₀-DSPE inside the outer layer of LEH was monitored by the assay reported earlier (Shimada et al., Int. J. Pharma., 203, 255-263, 2000). Approximately 28% of the added PEG₅₀₀₀-DSPE was incorporated into the bilayer. In order to convert CO-Hb back to O₂-Hb, the PEGylated LEHs as well as non-PEGylated LEHs were exposed to bright visible light from a 500 W halogen lamp under saturating oxygen atmosphere at 4-8° C. (Sakai et al., Bioconj. Chem., 11, 425-432, 1996). To concentrate, the preparations were centrifuged in a Beckman LE-80L ultracentrifuge at 184,000×g for 45 min to obtain LEH pellets. The pellets were washed two times with PBS (pH 7.4) and finally, resuspended in 300 mM sucrose in PBS (pH 7.4).

Characterization of LEH: The phospholipid concentration of the liposomes was determined by the method of Stewart (Stewart, Anal. Biochem., 104, 10, 1980). The oxygen affinity (p50) of encapsulated hemoglobin was measured on a Hemox-analyzer (TCS Scientific Corp., New Hope, Pa.). Amount of hemoglobin in LEHs was measured by monitoring absorbance of the OBG lysate of LEHs at 540 nm (Tomita et al., J. Nara Med. Assoc., 19, 1-6, 1968). Methemoglobin content of LEHs did not increase significantly above 3.7% (Matsuoka, Biol. Pharm. Bull., 20, 1208-1211, 1997). The size of the liposomes was determined by photon correlation spectroscopy using a Brookhaven particle size analyzer equipped with argon laser, BI-9000AT digital correlator and BI-200SM goniometer (Holtsville, N.Y.). Each sample was sized for 2 min with detector at 90° angle and sample housed in a 25° C. bath. The data was analyzed by non-negatively constrained least squares (CONTIN) using dynamic light scattering software-9KDLSW, beta version 1.24 supplied with the instrument.

Radiolabeling of LEH: LEHs were labeled essentially by the method developed by Phillips et al., (Phillips et al., J. Pharmacol. Exp. Ther., 288, 665-670, 1992). LEHs (1 ml) were mixed with 1 ml of ^{99m}Tc-hexamethyl propylene amine oxime (HMPAO) that was prepared by reconstituting the HMPAO kit (Ceretec, Nycomed-Amersham, Arlington Heights, Ill.) with 15 mCi of sodium ^{99m}Tc-pertechnetate in 5 ml of normal saline. After 30 minutes of incubation at room temperature, the LEHs were passed through a PD-10 column (Pharmacia Biotech, Sweden) to separate any radioactivity that was not associated with the LEH. Labeling efficiency was determined by counting LEHs before and after passing them through the column. Both PEGylated and non-PEGylated LEHs labeled with similar efficiency. Also, negligible loss of labeling efficiency was observed during the study when the LEH preparations were stored at 4-8° C.

The physical characteristics of the preparations are shown in Table 1. All the preparations were comparable in their size distribution, lipid content, p50, etc. Size of neutral LEH appeared to increase with time and therefore, its average size is significantly more than the other three preparations. The LEH's were labeled with ^{99m}Tc to monitor their distribution by gamma camera imaging and counting tissue-associated radioactivity on necropsy.

TABLE 1
Properties of LEHs injected in rabbits.

| | Size | [Lipid] | Lipid injected | ^{99m}Tc Labeling | | [Hb] | [Hb]/ |
|---|---|---|---|---|---|---|---|
| LEH | (nm ± sem) | mg/ml | per animal (mg) | Efficiency | p50 | g/dL | [Lipid] |
| Neutral | 266.6 ± 35.5 | 27.44 | 17.95 ± 1.4 | 85.63 ± 1.22% | 25.45 | | |
| PEG- | 189.8 ± 20.3 | 28.72 | 18.57 ± 2.3 | 86.35 ± 1.85% | 25.94 | 3.95 | 1.4 |
| Neutral | | | | | | | |
| Anionic | 151.2 ± 17.7 | 29.44 | 14.58 ± 1.6 | 85.20 ± 4.68% | 21.91 | | |
| PEG- | 135.7 ± 5.4 | 29.63 | 17.72 ± 0.7 | 76.00 ± 2.94% | 21.42 | 4.50 | 1.5 |
| Anionic | | | | | | | |

Animal Biodistribution and Imaging Studies: The animal experiments were performed according to the NIH Animal Use and Care Guidelines and were approved by the Institutional Animal Care Committee of the University of Texas Health Science Center at San Antonio. Male New Zealand white rabbits (n=4 per LEH preparation), weighing 2.5-3.0 Kg, were anesthetized by intramuscular injection of ketamine/xylazine mixture (50 and 10 mg/Kg body weight, respectively). Patency of arterial and venous lines was established by an angiocath and a butterfly, respectively. The ^{99m}Tc-LEHs were administered in 2 ml volume; lipid dose and radioactivity injected are given in Table 1. After intravenous administration of ^{99m}Tc-LEH, anterior whole body scintigrams (64×64 matrix) of the rabbits were acquired using a Picker Model Dyna 4 Gamma Camera (Cleveland, Ohio) interfaced to a Pinnacle computer (Medasys, Miami, Fla.). A low energy high-resolution collimator was used and the camera was peaked at 140 KeV with ±20% window. Arterial blood samples (100 μl) were obtained at various times after LEH injection. After imaging at 24 h the rabbits were euthanized by an overdose of an euthanasia solution (Buthenesia, Veterinary Labs, Inc., Lenexa, Kans.). Various organs were excised, washed with saline, weighed and appropriate tissue samples were counted in a gamma counter (Perkin-Elmer, Connecticut). Femur with bone marrow was taken as representative of bone. Total blood volume, bone and muscle mass were estimated as 5.4%, 10% and 40% of body weight, respectively (Frank, Physiological Data of Laboratory Animals, in “Handbook of Laboratory Animal Science” (Melby EC, Jr. ed) pp 23-64, CRC Press, Boca Raton, Fla.; Petty, “Research Techniques in the Rats,” Charles C. Thomas, Springfield, Ill.). A diluted sample of injected LEHs served as a standard for comparison.

Data Analysis: All average values are given±standard error of mean. The data was statistically analyzed by the univariate analysis of variance using SPSS software for Windows (Upper Saddle River, N.J.). The acceptable probability for significance was p<0.05. To determine the T_{1/2} of circulation, the circulation data was analyzed by the method of residuals. For quantitative analysis of scintiimages, regions of interest (ROI) were drawn around the organs of interest and normalized with the total number of counts registered in the image; the results were expressed as percent of whole body.

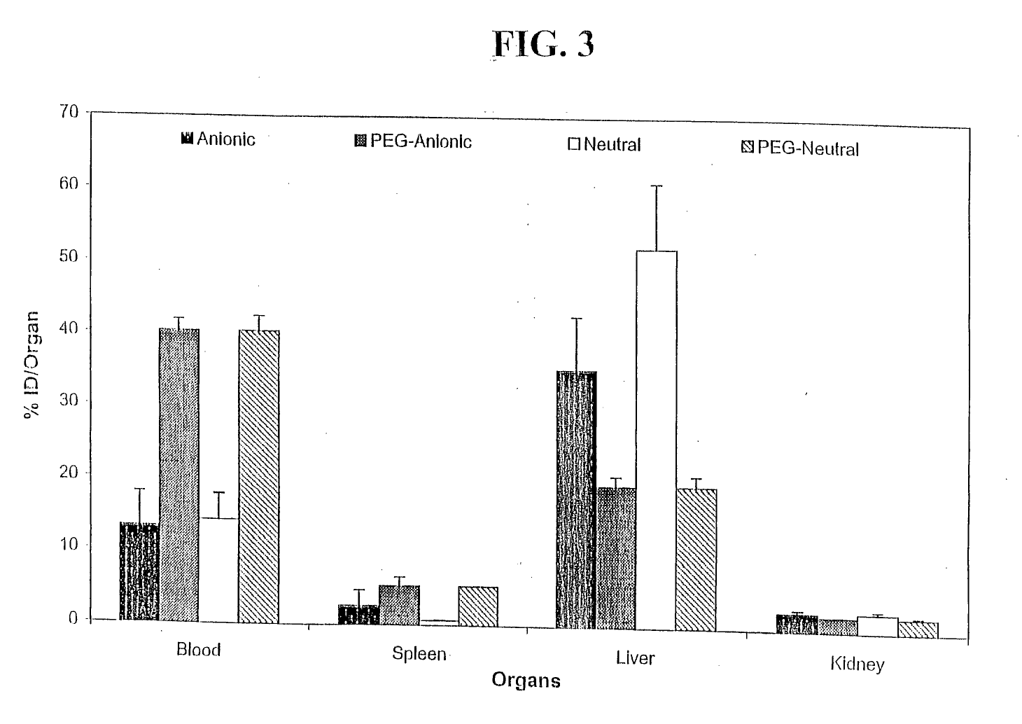

The accumulation of ^{99m}Tc-LEHs in various organs of rabbits is shown in Table 2. The major organs of accumulation of radioactivity were blood, spleen and liver (FIG. 3); other organs accumulated negligible amount of activity. The 24 h blood activity showed that PEGylated LEHs, both neutral and anionic, had prolonged circulation in blood with PEG-neutral LEH circulating slightly better than the PEG-anionic LEH. On the other hand, the non-PEGylated neutral and anionic LEHs circulated to almost the same extent (13-14% in blood at 24 h). Spleen activity was significantly lower in rabbits injected with non-PEGylated LEHs compared with PEGylated LEHs. It appears that PEGylation abolishes the effect of charge on spleen uptake that was found significantly different between neutral and anionic LEHs (neutral<anionic, p<0.05). Liver accumulated majority of activity in case of neutral (52.13%) and anionic (35.3%) LEHs. In contrast, PEGylated LEHs accumulated to the extent of 19% (PEG-neutral) and 12% (PEG-anionic) in liver. Similar pattern was observed in kidneys. Two other organs of significant accumulation were muscle and skin and both appear to follow the pattern shown by blood-borne activity. This was clear by the total recovered activity (>70% for neutral LEHs compared to about 60% for anionic LEHs). While there was a difference in spleen and liver uptake of neutral and anionic LEHs, PEGylation nullified the influence of charge on accumulation in blood, spleen and liver (FIG. 3).

TABLE 2(a)
Biodistribution of LEH preparations in rabbits (% ID per organ).

| | Neutral | PEG-Neutral | Anionic | PEG-Anionic |
|---|---|---|---|---|
| Blood | 13.99 ± 3.59*^{,}* | 40.27 ± 2.14 | 13.12 ± 4.73*^{,}** | 35.70 ± 1.63 |
| Spleen | 00.61 ± 0.10*^{,}* | 05.21 ± 0.07 | 02.56 ± 2.06*^{,}** | 06.31 ± 1.21 |
| Liver | 52.13 ± 8.93*^{,}* | 19.12 ± 1.47 | 35 26 ± 7.44*^{,}** | 11.51 ± 1.38 |
| Kidney | 02.40 ± 0.42 | 01.85 ± 0.24 | 02.38 ± 0.37 | 01.61 ± 0.05 |
| Lung | 00.33 ± 0.03 | 00.88 ± 0.06 | 00.49 ± 0.07 | 00.62 ± 0.03 |
| Heart | 00.06 ± 0.01 | 00.14 ± 0.02 | 00.05 ± 0.01 | 00.11 ± 0.00 |
| Muscle | 01.74 ± 0.33 | 03.87 ± 0.54 | 01.28 ± 0.25 | 01.89 ± 0.03 |
| Femur | 00.35 ± 0.02 | 00.38 ± 0.04 | 00.46 ± 0.07 | 00.36 ± 0.02 |
| Skin | 02.22 ± 0.51 | 04.27 ± 1.03 | 01.15 ± 0.11 | 02.09 ± 0.24 |
| Testis | 00.14 ± 0.03 | 00.31 ± 0.06 | 00.14 ± 0.05 | 00.22 ± 0.02 |
| Brain | 00.02 ± 0.01 | 00.04 ± 0.01 | 00.02 ± 0.00 | 00.04 ± 0.00 |
| Recovery | 73.99 ± 6.13 | 76.35 ± 5.23 | 56.91 ± 3.15 | 60.21 ± 2.16 |

* = p < 0.05 versus PEG-Neutral
** = p < 0.05 versus PEG-Anionic

TABLE 2(b)
Biodistribution of LEH preparations in rabbits (% ID per gram tissue).

| | Neutral | PEG-Neutral | Anionic | PEG-Anionic |
|---|---|---|---|---|
| Blood | 0.08 ± 0.02*^{,}** | 0.24 ± 0.02 | 0.09 ± 0.03*^{,}** | 0.23 ± 0.03 |
| Spleen | 0.64 ± 0.06*^{,}** | 4.99 ± 0.22 | 1.77 ± 1.14*^{,}** | 5.79 ± 0.76 |
| Liver | 0.54 ± 0.10*^{,}** | 0.19 ± 0.03 | 0.46 ± 0.01 | 0.16 ± 0.02 |
| Kidney | 0.13 ± 0.02 | 0.10 ± 0.01 | 0.14 ± 0.10 | 0.11 ± 0.01 |
| Lung | 0.04 ± 0.00 | 0.09 ± 0.01 | 0.05 ± 0.01 | 0.07 ± 0.00 |
| Heart | 0.01 ± 0.00 | 0.03 ± 0.00 | 0.01 ± 0.00 | 0.02 ± 0.00 |
| Muscle | 0.00 ± 0.00 | 0.00 ± 0.00 | 0.00 ± 0.00 | 0.00 ± 0.00 |
| Femur | 0.00 ± 0.00 | 0.00 ± 0.00 | 0.00 ± 0.00 | 0.00 ± 0.00 |
| Skin | 0.01 ± 0.00 | 0.01 ± 0.00 | 0.00 ± 0.00 | 0.01 ± 0.00 |
| Testis | 0.04 ± 0.01 | 0.07 ± 0.02 | 0.02 ± 0.00 | 0.04 ± 0.01 |
| Brain | 0.00 ± 0.00 | 0.01 ± 0.00 | 0.00 ± 0.00 | 0.01 ± 0.00 |

* = p < 0.05 versus PEG-Neutral
** = p < 0.05 versus PEG-Anionic

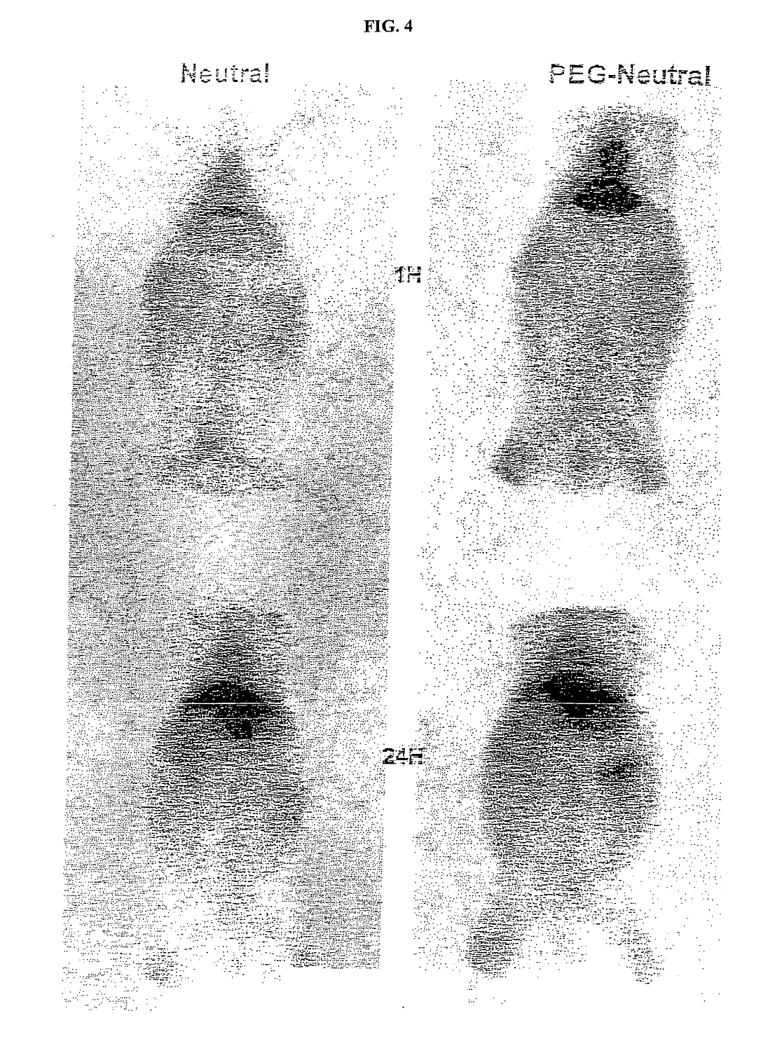

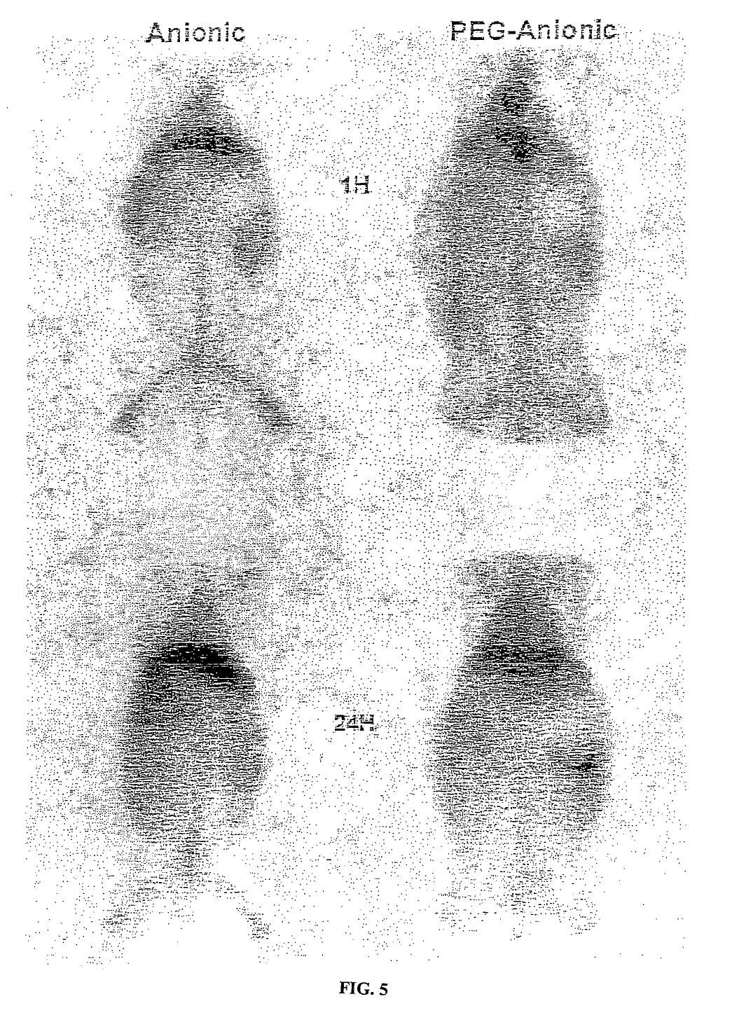

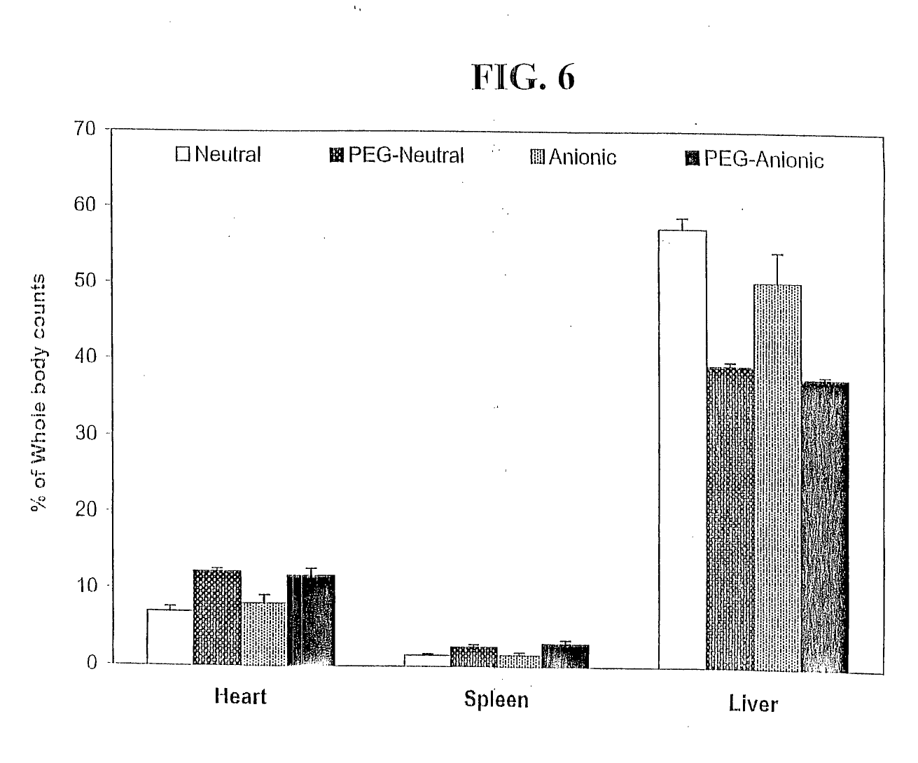

One advantage of using gamma ray emitting radionuclide (^{99m}Tc) was the capability of imaging the distribution of LEH in vivo without sacrificing the animal. FIGS. 4 and 5 show the early (1 h) and late (24 h) images of rabbits after injection of ^{99m}Tc-LEH. The scintigraphic images provided essentially the same information that was obtained by sacrificing the animal and counting various organs for radioactivity. Circulating activity in the images was estimated by evaluating the activity seen in the heart (FIGS. 4 and 5). Early images demonstrate rapidly diminishing heart activity when non-PEGylated LEHs were injected (FIGS. 4 and 5, left panels). On ROI analysis it was found that at 1 h, neutral LEH was approximately 7% of whole body activity while anionic LEH was about 8.1% (FIG. 6). The corresponding values for PEGylated LEHs were 12.2% and 11.7%, respectively. Apparently, at 1 h, the liver uptake of non-PEGylated LEHs was already exceeding that of PEGylated LEHs, although blood pool activity partially contributes to the apparent liver uptake in images (FIG. 6). By 24 h, the images of animals injected with non-PEGylated LEHs were characterized by high liver uptake and negligible heart activity. PEGylation, on the other hand, substantially enhanced the blood borne activity. Again, as was observed in tissue distribution studies, PEGylation increased LEH accumulation in spleen, but reduced that in liver. Little bladder activity that showed up in the 24 h images was due to the excretion of hydrophilic ^{99m}Tc-chelates after metabolic degradation of liposome structure and ^{99m}Tc-HMPAO. From this data it is evident that post-inserted PEG-neutral LEH has significantly longer circulation T_{1/2} than other preparations.

Of the major organs of liposome accumulation, accumulation in spleen is dependent largely on the PEGylation state of LEH (FIG. 6) and partially on the charge of the liposomes. On the other hand, liver uptake and circulating activity appear to be inversely related (Table 2a). Since liposomes without charge have a tendency to coalesce and increase in size on storage (Table 1), neutral liposomes without PEG-DSPE appear to accumulate in liver more than the anionic LEH without PEG-DSPE. Rapid metabolic turnover of anionic lipid in the RES might also be the reason of ^{99m}Tc-neutral liposomes being recovered more than the ^{99m}Tc-anionic liposomes (Table 2a).

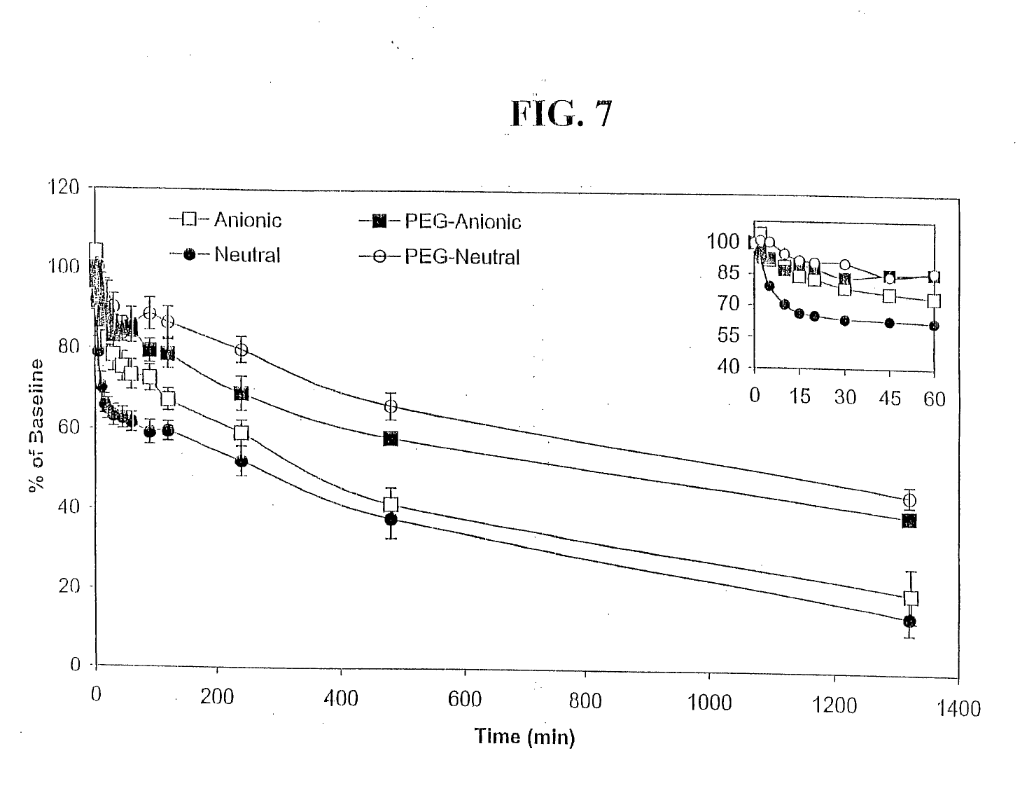

Simultaneous to the dynamic and static image acquisition of animals, blood samples were withdrawn at intermittent times during the 24 h period of study. These samples were counted for circulating radioactivity. FIG. 7 shows the circulation profiles of the LEH preparations in blood. The amounts of radioactivity still circulating at 24 h were 14.4, 20.1, 44.8 and 39.5% for neutral, anionic, PEG-neutral and PEG-anionic LEH, respectively. The 24 h arterial data corroborated very closely with blood borne activity from tissue distribution data described above. All the preparations seemed to drop from circulation in a biphasic pattern. Compared to PEGylated LEHs, the first phase in case of non-PEGylated LEHs was steep. There was minimal difference among PEG-neutral, PEG-anionic and anionic LEHs during the first hour of injection, but neutral LEH started showing significantly less circulating activity as early as 5 min (inset, FIG. 7). More than 50% of neutral or anionic LEH disappeared from circulation within 6 h of injection, whereas the PEGylated LEHs achieved the same level only after 15-20 h. The estimated T_{1/2} of neutral, anionic, PEG-neutral and PEG-anionic LEHs were 8.9, 9.6, 19.3 and 16.5 h, respectively.

It is also clear that a post-insertion compound improves circulation half-life of LEH. Since the reported half-life of circulation is comparable to that reported previously for a small dose of liposomes (Goins et al., J. Nucl. Med., 37, 1374-1379, 1996), it is apparent that the inserted PEG-DSPE is not lost in circulation.

### Example 2

### Kinetics of ^{99m}Tc-PEG-Neutral LEH in Rat and Rabbit Models of 25% Exchange Transfusion

Preparation of LEH: The liposomes were prepared as described above in Example 1.

Recycling of unencapsulated hemoglobin was used in the manufacturing of LEH. The unencapsulated hemoglobin was collected as filtrate during 500 KDa (MWCO) ultrafiltration step (FIG. 2) and concentrated by another ultrafiltration step (10 KDa MWCO) for use in subsequent batches of LEH. This recycling was performed at least 3 times without any changes in oxygen carrying property or methemoglobin formation. The LEH preparation was a combined mixture of all three LEH batches made out of recycled hemoglobin. During LEH manufacturing and hemoglobin recycling, hemoglobin was in carbonyl hemoglobin form that stabilizes hemoglobin against temperature-sensitive degradation.

Characterization of LEHs: The liposomes were characterized as described above in Example 1.

Radiolabeling of LEH: The LEH were labeled as described above in Example 1.

The results of the characterization of the liposomes are shown in Table 3.

TABLE 3
Properties of LEH.

| | | | | | Colloidal | | |
|---|---|---|---|---|---|---|---|
| | | | | | oncotic | % PEG- | ^{99m}Tc |
| | Size | [Hb] | [Lipid] | Osmolality | pressure | DSPE | Labeling |
| p50 | (nm ± sem) | (g/dL) | (mg/ml) | (mOsmol/kg) | (mm Hg) | insertion | Efficiency |
| 19.9 | 133.1 ± 31.7 | 3.4 | 59 | 320 | 20.2 | 52.14% | 77% |

Animal Biodistribution and Imaging Studies: The animal experiments were performed according to the NIH Animal Use and Care Guidelines and were approved by the Institutional Animal Care Committee of the University of Texas Health Science Center at San Antonio.

Rat exchange model: The rat exchange model has been described earlier (Goins et al., Shock, 4, 121-130, 1995). Left femoral artery of male Sprague Dawley rats (350-450 g) was cannulated with polyethylene tubing filled with heparin and subcutaneously tunneled to the back. After closing the surgical area, the rats were given 2 days to recover from the procedure. On the day of the exchange, the rats were anesthetized with isoflurane gas and 25% of blood volume was withdrawn through the tubing at the rate of 0.5 ml/min. The tubing was filled with heparin again and after giving 10 min time for equilibration, ^{99m}Tc-LEH (equal to the amount of blood withdrawn) was infused through the tail vein (0.5 ml/min). Total volume of blood was estimated as 5.7% of body weight. Blood samples (50 μl) were withdrawn at various times through the tubing for counting of blood borne radioactivity. Dynamic gamma camera images were acquired for 30 min after the start of transfusion and static images were acquired at 4, 24 and 48 h of infusion. Images were acquired with matrix size of 256×256 for time sufficient to obtain significant radioactive counts. Further details of the imaging are discussed below.

Rabbit exchange model: Male New Zealand white rabbits (n=3), weighing 2.0-2.5 Kg, were anesthetized by intramuscular injection of ketamine/xylazine mixture (50 and 10 mg/Kg body weight, respectively). Patency of arterial and venous lines was established by an angiocath and a butterfly, respectively. Total 25% of circulating blood was withdrawn through arterial line (0.5 ml/min) and animals were given 10 min to equilibrate before infusing equal volume of ^{99m}Tc-LEHs through venous line. After intravenous administration of ^{99m}Tc-LEH, anterior whole body scintigrams (64×64 matrix) of the rabbits were acquired using a Picker Model Dyna 4 Gamma Camera (Cleveland, Ohio) interfaced to a Pinnacle computer (Medasys, Miami, Fla.). A low energy high-resolution collimator was used and the camera was peaked at 140 KeV with +20% window. Arterial blood samples (100 μl) were obtained at various times after LEH injection.

About 25% of estimated circulating blood was exchanged with LEH without any apparent distress to the animals (rats, n=7 and rabbits n=3).

After imaging at 48 h the animals were euthanized by an overdose of an euthanasia solution (Buthenesia, Veterinary Labs, Inc., Lenexa, Kans.). Various organs were excised, washed with saline, weighed and appropriate tissue samples were counted in a gamma counter (Perkin-Elmer, Connecticut). Total blood volume and muscle mass were estimated as 5.7% and 40% of body weight, respectively (Frank, 1976; Petty, 1982). A diluted sample of injected LEHs served as a standard for comparison.

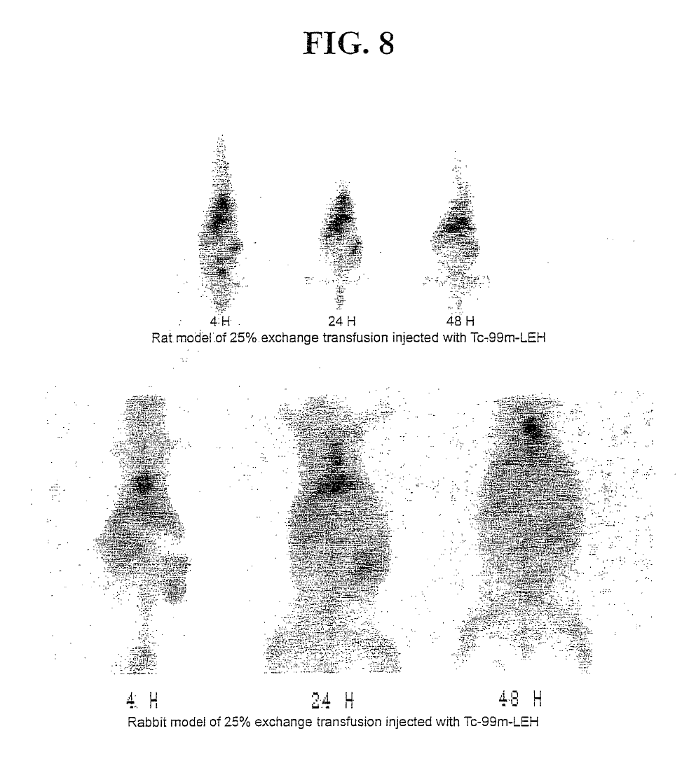

FIG. 8 shows a representative set of gamma camera acquired rat and rabbit images. Circulating LEH in animals can be estimated by amount of radioactivity in heart. It is clear that even after 48 h significant amount of LEH was still in blood—a property necessary of a long-acting oxygen carrier. Evidently, the long-circulation of LEH was the result of post-inserted PEG-DSPE.

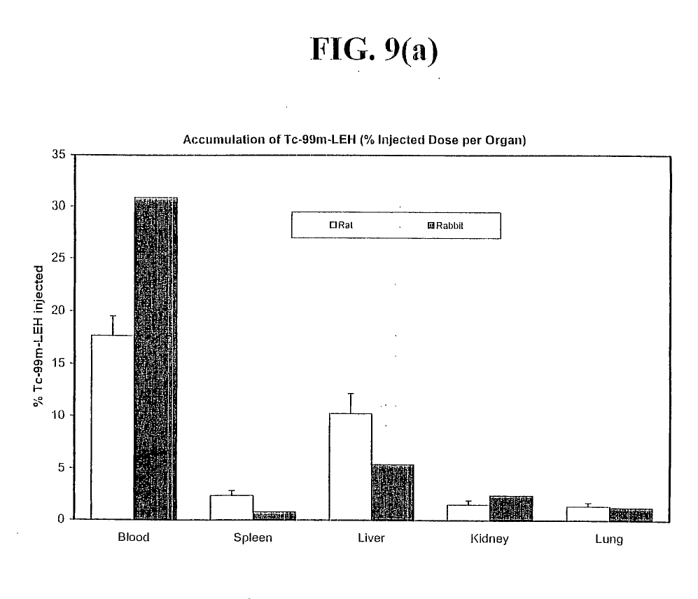

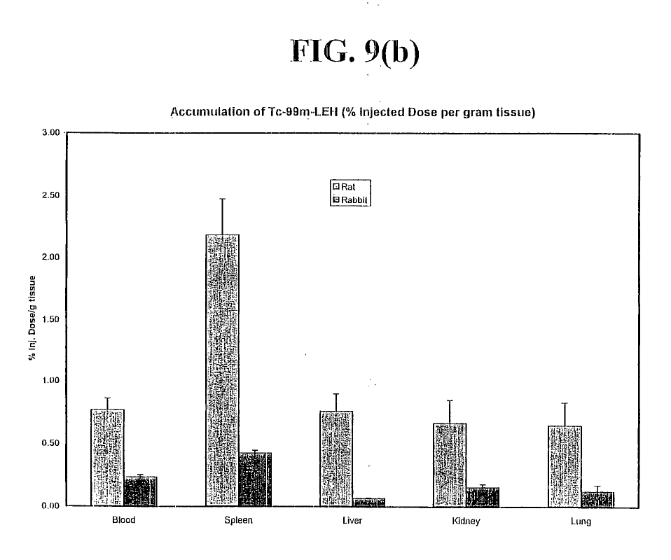

The amount of LEH accumulated in various organs of rats and rabbits after 48 h of infusion is shown in Table 4. The major organs of accumulation of radioactivity were blood, spleen and liver in both rats and rabbits (FIG. 9); other organs accumulated negligible amount of activity. There were some significant differences between rats and rabbits in terms of the extent of accumulation of LEH in blood, spleen and liver. Rat liver and spleen accumulated more LEH than rabbit liver and spleen. Correspondingly, blood borne LEH was lesser in rats than in rabbits (FIG. 9a), but on per gram tissue basis the amount of LEH in rat blood was much more than that in rabbit blood (FIG. 9b). In fact, all the organs in rats accumulated considerably more than the rabbit on per gram tissue basis (FIG. 9b). Two other organs of significant accumulation were muscle and skin and both appeared to follow the pattern shown by blood-borne activity.

TABLE 4
Accumulation of ^{99m}Tc-LEH in various organs of rats
and rabbits after 25% exchange transfusion.

| | Rats | | | | Rabbits | | | |
|---|---|---|---|---|---|---|---|---|
| | Mean | SEM | Mean | SEM | Mean | SEM | Mean | SEM |
| Organ | ID/g tissue | | ID/Organ | | ID/g tissue | | ID/Organ | |
| Blood | 0.77 | 0.09 | 17.65 | 1.87 | 0.24 | 0.02 | 30.88 | 0.38 |
| Spleen | 2.18 | 0.29 | 2.39 | 0.46 | 0.43 | 0.02 | 0.78 | 0.14 |
| Liver | 0.75 | 0.14 | 10.25 | 1.92 | 0.07 | 0.00 | 5.42 | 0.43 |
| Kidney | 0.66 | 0.18 | 1.53 | 0.40 | 0.16 | 0.02 | 2.42 | 0.28 |
| Lung | 0.65 | 0.18 | 1.37 | 0.37 | 0.13 | 0.05 | 1.25 | 0.28 |
| Heart | 0.36 | 0.20 | 0.43 | 0.23 | 0.03 | 0.00 | 0.16 | 0.01 |
| Muscle | 0.01 | 0.00 | 1.26 | 0.34 | 0.00 | 0.00 | 2.50 | 0.73 |
| Bowel + | 0.19 | 0.04 | 3.84 | 0.93 | ND | ND | ND | ND |
| Stomach | | | | | | | | |
| Skin | ND* | ND | ND | ND | 0.00 | 0.00 | 1.22 | 0.41 |

*Not determined.

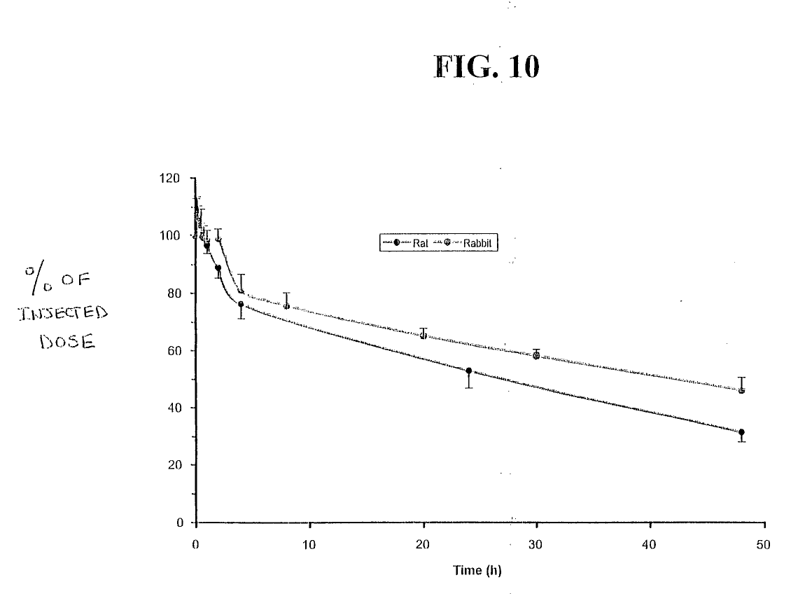

Simultaneous to the gamma camera imaging of animals, blood samples were withdrawn at intermittent times during the 48 h period of study. These samples were counted for circulating radioactivity. FIG. 10 shows the circulation profiles of the LEH preparations in blood of both rats and rabbits. The amounts of radioactivity still circulating at 48 h were 31% for rats and 46% for rabbits. The 48 h arterial data corroborated very closely with blood borne activity from tissue distribution data described above. LEH circulation in both rats and rabbits followed a biphasic pattern; that is, a rapid decrease during first 4 h followed by a more gradual drop during the rest of the study. The estimated T_{1/2} of LEH circulation after 25% exchange transfusion was about 30 h in rats and 39.8 h in rabbits, respectively.

The data shows that unencapsulated hemoglobin in carbonylhemoglobin form can be recycled at least 3 times. Also, exchange of rodent's blood with PEG-neutral LEH up to 25% circulating volume can be done without any apparent problem.

### Example 3

### Effect of PEGylation on Thrombocytopenic Effect-Induced of Neutral and Anionic LEHs

Platelet Studies: Intravenous administration of liposomes can induce a sudden and rapid drop in circulating platelets that recover over time. The extent of thrombocytopenia and the time for recovery depend upon the method of administration (bolus or slow), the amount administered, composition of liposomes and any associated impurity. In general, rapid injection has more severe effect as compared to slow infusion. However, lipid composition of liposomes overrides the other factors in terms of degree of thrombocytopenia observed. Thus, liposomes containing negatively charged (anionic) lipids can induce a very rapid platelet drop that is more severe and that takes a longer time to recover to normal levels. This effect of charge is partially prevented by coating liposome surface with lipid-linked hydrophilic polymers, such as polyethylene glycol (PEG).

The problem of platelet-effect is a major concern in developing liposome-encapsulated hemoglobin (LEH) as an oxygen carrier. The indications for use of LEH would include situations of heavy blood loss and usually a patient is in a very critical stage. LEH-induced thrombocytopenia is not a tolerable phenomenon in such cases. Therefore, lipid composition of LEH should be chosen in such a fashion as to reduce this effect.

The following four types of LEHs were manufactured as described above: (1) Neutral LEH (DSPC/Cholesterol/α-tocopherol, 51.4:46.4:2.2); (2) PEG-Neutral LEH (DSPC/Cholesterol/α-tocopherol, 51.4:46.4:2.2) post-inserted with PEG-DSPE; (3) Anionic LEH (DSPC/Chol/DMPG/α-tocopherol, 46:42:9.8:2.2); and (4) PEG-Anionic LEH (DSPC/Chol/DMPG/α-tocopherol, 46:42:9.8:2.2) post-inserted with PEG-DSPE.

In-111 radiolabeling of rabbits platelets: New Zealand white Rabbits (2.5 Kg) were anesthetized with ketamine/xylazine. About 40 ml of blood was withdrawn via central ear artery and processed to separate pure population of platelets. The recovered platelets were labeled with In-111-oxine (100 microCi) by the method reported elsewhere (Thakuv, Thrombosis Rev., 9, 345-357, 1976), while the separated red blood cell fraction was re-infused back into the animal.

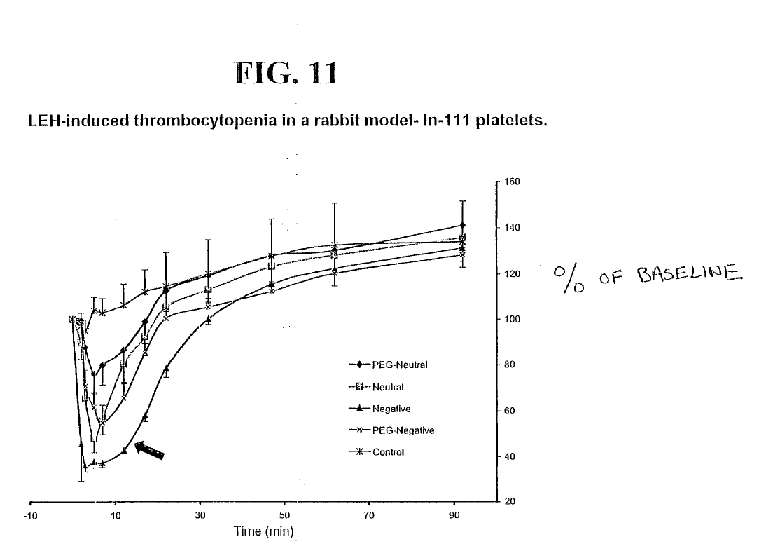

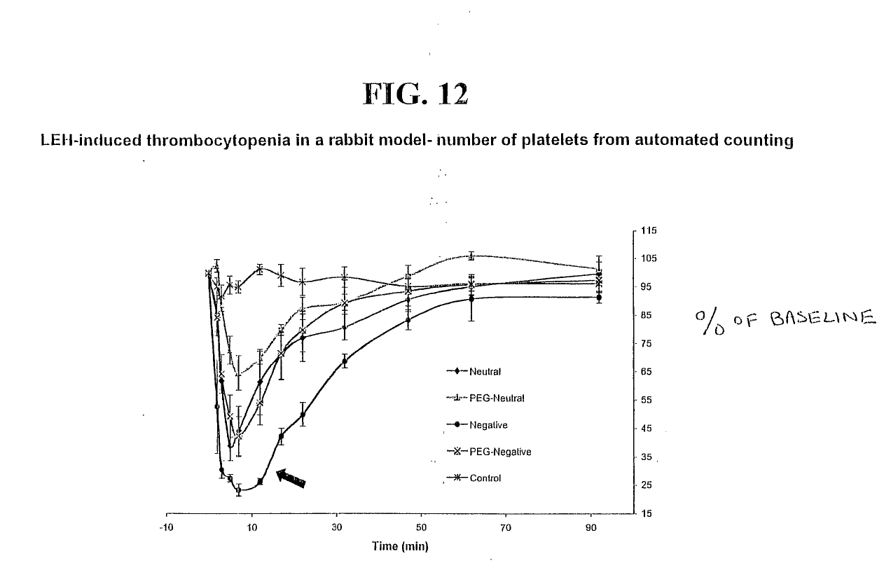

Animal Study: In-111-platelets (30-70 microCi) were suspended in about 5 ml of saline and infused over 2 min via marginal ear vein. After allowing 30 min for platelets to attain equilibrium, a small dose of LEH (1 ml, about 10 mg phospholipid) was injected intravenously over a period of 1 min. Samples of blood (about 0.4 ml) were withdrawn through an arterial catheter during a period of 2 h after In-111-platelet injection. The samples of blood were taken for radioactivity counting in a gamma counter (FIG. 11) as well as automated complete blood cell counting (FIG. 12).

FIG. 11 shows the circulating radiolabeled platelets after LEH injection. All LEH preparations of LEH induced rapid and transient reduction (arrow) in the circulating In-111-platelets. Compared to control saline injection, the effect was very severe in case of anionic LEH and was minimal in PEG-neutral LEH. Over 60% of In-111-platelets went out of circulation within 5 minutes of anionic LEH injection, accompanied by obvious breathing discomfort and dyspnea in the animal. On PEGylation of anionic LEH the platelet drop was reduced (˜50%) and the apparent breathing abnormalities were abrogated. The platelet recovery was also faster. Neutral LEH behaved in a way similar to PEG-anionic LEH; the percentage drop of circulating platelets and the recovery were about the same. However, PEG-neutral LEH had considerably reduced thrombocytopenic effect, with very fast recovery. Only about 20% circulating platelets dropped from circulation and the recovered within 25-30 minutes. No breathing abnormalities were observed with either the neutral or the PEG-neutral LEH.

Simultaneous to the radioactivity counting, a fraction of withdrawn blood was sent for automated complete blood cell counting. The results corroborated very well with the data obtained from radioactivity counting (FIG. 12). Again PEG-neutral LEH was the least thrombocytopenic compared with the other preparations.

Throughout this application, various publications are referenced. The disclosures of these publications in their entireties are hereby incorporated by reference into this application in order to more fully describe the state of the art to which the disclosed subject matter pertains.

It will be apparent to those skilled in the art that various modifications and variations can be made in the disclosed materials and methods without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A neutral liposome comprising an encapsulated compound and a post-insertion compound, wherein the post-insertion compound comprises a hydrophilic component and an anchoring component, wherein the encapsulated compound is located within the neutral liposome, and the post-insertion compound is adjacent to the outer surface of the neutral liposome.

2. The liposome of claim 1, wherein the liposome comprises one or more neutral lipids.

3. The liposome of claim 2, wherein the neutral lipid comprises phosphatidyl choline, sphingomyelin, dipalmitoyl phosphatidylcholine, or hydrogenated soy phosphatidylcholine.

4. The liposome of claim 2, wherein the neutral lipid comprises distearoyl phosphatidylcholine.

5. The liposome of claim 1, wherein the neutral liposome further comprises a steroid compound, an anti-oxidant, or a combination thereof.

6. The liposome of claim 1, wherein the neutral liposome further comprises an antioxidant, and the antioxidant comprises glutathione or homocysteine.

7. The liposome of claim 1, wherein the neutral liposome further comprises a steroid compound, and the steroid compound comprises cholestanol, coprostanol, cholestane, or an organic acid derivative of a sterol.

8. The liposome of claim 1, wherein the neutral liposome further comprises a steroid compound, and the steroid compound is cholesterol.

9. The liposome of claim 1, wherein the neutral liposome contains no anionic lipid.

10. The liposome of claim 1, wherein the neutral liposome contains one or more anionic lipids, wherein the total amount of the anionic lipids is less than 6 mole percent of the total lipids.

11. The liposome of claim 10, wherein the anionic lipid comprises of phosphatidyl serine, phosphatidyl inositol, phosphatidic acid, cardiolipin, or phosphatidyl glycerol.

12. The liposome of claim 10, wherein the anionic lipid comprises dimyristoyl phosphatidylglycerol.

13. The liposome of claim 1, wherein the encapsulated compound comprises hemoglobin, a protein, an enzyme, an immunoglobulin, a peptide, an oligonucleotide, or a nucleic acid.

14. The liposome of claim 1, wherein the encapsulated compound comprises hemoglobin, wherein the hemoglobin comprises stroma-free hemoglobin.

15. The liposome of claim 14, wherein the amount of hemoglobin that is encapsulated within the liposome is from 1 to 12 g/dl.

16. The liposome of claim 1, wherein the post-insertion compound comprises the reaction product between a hydrophilic compound and an anchoring compound.

17. The liposome of claim 16, wherein the hydrophilic compound comprises polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethylene glycol, polyaspartamide, or a hydrophilic peptide sequence.

18. The liposome of claim 16, wherein the hydrophilic compound comprises polyethylene glycol.

19. The liposome of claim 16, wherein the anchoring compound comprises phosphatidylethanolamine with a fatty acid chain having from 14 to 22 carbon atoms, cholesterol, or ceramide.

20. The liposome of claim 1, wherein the post-insertion compound comprises polyethylene glycol-distearoyl phosphatidylethanolamine.

21. The liposome of claim 1, wherein the liposome further comprises a plasma expander.

22. The liposome of claim 21, wherein the plasma expander comprises a starch compound, albumin, dextran, or gelatin.

23. The liposome of claim 21, wherein the plasma expander comprises hetastarch or hydroxyethyl starch.

24. The liposome of claim 21, wherein the plasma expander comprises pentastarch.

25. The liposome of claim 1, wherein the size of the liposome is from 100 nm to 350 nm.

26. The liposome of claim 1, wherein the size of the liposome is from 200 nm to 275 nm.

27. The liposome of claim 1, wherein the liposome is composed of distearoyl phosphatidylcholine, the encapsulated compound is stroma-free hemoglobin, the post-insertion compound is polyethylene glycol-distearoyl phosphatidylethanolamine, and the liposome further comprises pentastarch.

28. A pharmaceutical composition comprising the liposome of claim 1 and a pharmaceutically-acceptable carrier.

29. A method for preparing a liposome-encapsulated compound, comprising:
(a) admixing an unencapsulated compound with at least one neutral lipid;
(b) microfluidizing the suspension produced in step (a) to produce a mixture comprising a first liposome and unencapsulated compound;
(c) ultrafiltering the mixture produced in step (b) to remove the unencapsulated compound; and
(d) contacting the resultant liposomes after ultrafiltering step (c) with a post-insertion compound.

30. The method of claim 29, wherein a plasma expander is added after step (a) and prior to step (d).

31. The method of claim 29, wherein after step (b) and prior to step (c), the first liposome is contacted with a plasma expander.

32. The method of claim 29, wherein the method is continuous.

33. The method of claim 29, wherein the unencapsulated compound after step (c) is recycled and introduced into step (a).

34. The liposome produced by the method of claim 29.

35. A method of treating or preventing a disease in a subject comprising administering to the subject the liposome of claim 1.

36. A method for screening a liposome-encapsulated compound for an activity, comprising the steps of:
a) measuring a known activity or pharmacological activity of the liposome-encapsulated compound of claim 1; and
b) measuring the same activity or pharmacological activity of the corresponding unencapsulated compound.

37. A method of treating or preventing a disease in a subject comprising administering to the subject the pharmaceutical composition of claim 28.
